(19) United States
(12) Patent Application Publication
(10) Pub. No.: US 2024/0117035 A1
(43) Pub. Date: Apr. 11, 2024

(54) CLDN18.2/CD3 BISPECIFIC ANTIBODIES FOR THE THERAPY OF CLDN18.2-EXPRESSING SOLID TUMORS

(71) Applicant: Shandong Boan Biotechnology Co., Ltd., Yantai (CN)

(72) Inventors: Li ZHOU, Yantai (CN); Avanish VARSHNEY, Yantai (CN); Deyong SONG, Yantai (CN); Chuangchuang DONG, Yantai (CN); Ninghai WANG, Yantai (CN)

(21) Appl. No.: 18/276,159

(22) PCT Filed: Feb. 7, 2022
(86) PCT No.: PCT/CN2022/075326

§ 371 Date: Aug. 7, 2023

Related U.S. Application Data
(60) Provisional application No. 63/147,233, filed on Feb. 8, 2021.

(51) Int. Cl. C07K 16/28

(52) U.S. Cl. CPC C07K 16/28; C07K 16/2809; C07K 2317/24; C07K 2317/31; C07K 2317/622; C07K 2317/71; C07K 2317/73; C07K 2317/92

(57) ABSTRACT

The present application provides CLDN18.2 antibodies or antigen binding fragments thereof and related CLDN18.2/CD3 bispecific antibodies, as well as corresponding nucleic acids encoding same, vectors comprising such nucleic acids, host cells transfected with such nucleic acids or vectors. The present application further relates to isolated CLDN18.2 antibodies or antigen binding fragments thereof and related CLDN18.2/CD3 bispecific antibodies, pharmaceutical compositions comprising such antibodies, antigen binding fragments, bispecific antibodies, nucleic acids, vectors, or host cells and optionally a pharmaceutically acceptable carrier, and methods of treating cancer in a subject in need thereof by administering such pharmaceutical compositions. The cancers treated in accordance with the present application include CLDN18.2-positive cancers, such as, inter alia, gastric cancer and pancreatic cancer.

This application claims the benefit of U.S. Provisional Patent Application No. 63/147,233, entitled “CLDN18.2/CD3 bispecific antibodies for the therapy of CLDN18.2-expressing solid tumors”, filed on Feb. 8, 2021, the content of which is herein incorporated by reference in its entirety.

### TECHNICAL FIELD

The present application relates to CLDN18.2 antibodies or antigen binding fragments thereof and related CLDN18.2/CD3 bispecific antibodies, as well as corresponding nucleic acids encoding same, vectors comprising such nucleic acids, host cells transfected with such nucleic acids or vectors. The present application further relates to isolated CLDN18.2 antibodies or antigen binding fragments thereof and related CLDN18.2/CD3 bispecific antibodies, pharmaceutical compositions comprising such antibodies, antigen binding fragments, bispecific antibodies, nucleic acids, vectors, or host cells and optionally a pharmaceutically acceptable carrier, and methods of treating cancer in a subject in need thereof by administering such pharmaceutical compositions. The cancers treated in accordance with the present application include CLDN18.2-positive cancers, such as, inter alia, gastric cancer and pancreatic cancer.

### BACKGROUND

CLDN18.2 protein is a transmembrane protein, which belongs to the Claudins (CLDNs) family. The entire protein is expressed on the cell membrane and is an important structural component of the tight junction of cells. Claudins have 4 transmembrane regions, 2 extracellular loops and 1 intracytoplasmic loop, which participate in the formation of tight junctions between cells. There are two alleles in the first exon of human CLDN18 gene, which can express two different splicing mutants known as CLDN18.1 and CLDN18.2, resulting in a 69 amino acid sequence at the N-terminal including extracellular loop 1. Therefore, there are differences between the two extracellular epitopes. Although most CLDNs are widely expressed, individual members are often highly selectively expressed in specific tissues.

Among them, CLDN18.1 protein is a specific antigen selectively expressed by alveolar epithelial cells, which is only highly expressed in normal alveolar tissues, but not found in other normal tissues, including pancreatic ducts; CLDN18.2 protein is also a highly selective marker protein, but its distribution is completely different than CLDN18.1 protein. The expression of CLDN18.2 protein is highly restricted in normal healthy tissues. It is not expressed in undifferentiated gastric stem cells. It is only expressed in differentiated gastric mucosal membrane epithelial cells, and the expression level is very limited. This is conducive to maintaining the gastric mucosa, whose barrier function can prevent H+ in gastric acid from leaking through paracellular pathways. However, CLDN18.2 protein frequently undergoes abnormal changes during the development of a variety of malignant tumors. For example, when gastric epithelial tissue undergoes malignant transformation, the resultant disorder of cell polarity will cause the CLDN18.2 protein epitope on the cell surface to be exposed. At the same time, CLDN18.2 gene will also be abnormally activated, and highly selectively and stably expressed in specific tumor tissues involved in the proliferation, differentiation, and migration of tumor cells.

In recent years, CLDN18.2 has become a promising target for tumor therapy. So far, nearly 20 innovative pharmaceutical companies have deployed CLDN18.2-targeted drug development. Most of these research projects are single-target antibody drugs. Among them, CLDN18.2 specific antibody Claudiximab (Zolbetuximab/IMAB362) has achieved remarkable success in clinical trials. A phase II study from Europe showed that compared with chemotherapy alone, the overall survival time of patients with advanced gastric cancer was extended from 8.4 months to 13.2 months after treatment with IMAB362 and standard chemotherapy. In this study, patients with the highest claudin 18.2 levels had a longer median overall survival time (16.7 months). Moreover, CLDN18.2 double-antibody drugs have also ushered in exciting new developments in solid tumor therapy. Recently, a new CLDN18.2/CD3 bispecific antibody, AMG 910, has been approved for Phase I clinical trials for gastric cancer and gastroesophageal junction cancer.

Overall, the evidence from current clinical trials has demonstrated that CLDN18.2 antibodies have high safety, tolerability, and anti-tumor activity as a targeted drug for patients with CLDN18.2-positive advanced gastric cancer.

### SUMMARY OF THE INVENTION

The present application provides novel CLDN18.2 antibodies or antigen binding fragments thereof and also related bispecific antibodies with particularly advantageous properties such as high producibility, stability, binding affinity, biological activity, specific targeting of CLDN18.2-positive cells, targeting efficiency, remaining tumor cell killing and/or reduced toxicity.

In one aspect, the present application provides a CLDN18.2 antibody or antigen binding fragment thereof, comprising a heavy chain variable region and a light chain variable region, the heavy chain variable region comprises HCDR1 comprising the amino acid sequence represented by SEQ ID NO. 11, HCDR2 comprising the amino acid sequence represented by SEQ ID NO. 12 and HCDR3 comprising the amino acid sequence represented by SEQ ID NO. 13, and/or the light chain variable region comprises LCDR1 comprising the amino acid sequence represented by SEQ ID NO. 14, LCDR2 comprising the amino acid sequence represented by SEQ ID NO. 15 and LCDR3 comprising the amino acid sequence represented by SEQ ID NO. 16.

In another aspect of the present application, the antigen binding fragment is selected from scFv fragment, Fv fragment, F(ab′)2 fragment, Fab′-SH fragment and Fab′ fragment.

In a further aspect, the CLDN18.2 antibody or antigen binding fragment binds to CLDN 18.2, and does not bind to CLDN 18.1.

In a further aspect, the above-mentioned antibody is a monoclonal antibody or a humanized antibody.

In a further aspect, such monoclonal antibody or humanized antibody includes antibodies produced by hybridomas or host cells transformed with an expression vector modified to carry an antibody gene by genetic engineering techniques. In a further embodiment, the monoclonal antibody or humanized antibody may comprise IgG antibodies comprising two heavy chains and two light chains; heavy chains of mouse IgG antibodies are classified into five subclasses according to the constant region of their heavy chains: IgG1, IgG2a, IgG2b, IgG2c and IgG3, and the light chains may be mouse lambda or kappa type; and heavy chains of human IgG antibodies are further classified into four subclasses according to the constant region of their heavy chains: IgG1, IgG2, IgG3, and IgG4, and the light chains may be human lambda or kappa type.

In a further aspect, the disclosure also provides a monoclonal CLDN18.2 antibody including a light chain variable region and a heavy chain variable region, wherein the heavy chain variable region comprises an amino acid sequence with at least 95%, 96%, 97%, 98%, 99% or 100% sequence identity to the sequence of SEQ ID NO. 1, and/or the light chain variable region comprises an amino acid sequence with at least 95%, 96%, 97%, 98%, 99% or 100% sequence identity to the sequence of SEQ ID NO. 2.

In a further aspect, the disclosure also provides a monoclonal CLDN18.2 antibody including a light chain variable region and a heavy chain variable region, wherein the heavy chain variable region comprises an amino acid sequence with at least 95%, 96%, 97%, 98%, 99% or 100% sequence identity to the sequence of SEQ ID NO. 3, and/or the light chain variable region comprises an amino acid sequence with at least 95%, 96%, 97%, 98%, 99% or 100% sequence identity to the sequence of SEQ ID NO. 4.

In a further aspect, the disclosure also provides a monoclonal CLDN18.2 antibody including a light chain variable region and a heavy chain variable region, wherein the heavy chain variable region comprises an amino acid sequence with at least 95%, 96%, 97%, 98%, 99% or 100% sequence identity to the sequence of SEQ ID NO. 5, and/or the light chain variable region comprises an amino acid sequence with at least 95%, 96%, 97%, 98%, 99% or 100% sequence identity to the sequence of SEQ ID NO. 6.

In a further aspect, the disclosure also provides a monoclonal CLDN18.2 antibody including a light chain variable region and a heavy chain variable region, wherein the heavy chain variable region comprises an amino acid sequence with at least 95%, 96%, 97%, 98%, 99% or 100% sequence identity to the sequence of SEQ ID NO. 7, and/or the light chain variable region comprises an amino acid sequence with at least 95%, 96%, 97%, 98%, 99% or 100% sequence identity to the sequence of SEQ ID NO. 8.

In a further aspect, the disclosure also provides a monoclonal CLDN18.2 antibody including a light chain variable region and a heavy chain variable region, wherein the heavy chain variable region comprises an amino acid sequence with at least 95%, 96%, 97%, 98%, 99% or 100% sequence identity to the sequence of SEQ ID NO. 9, and/or the light chain variable region comprises an amino acid sequence with at least 95%, 96%, 97%, 98%, 99% or 100% sequence identity to the sequence of SEQ ID NO. 10.

In a further aspect, the antibody or antigen-binding fragment thereof is a humanized antibody or antigen-binding fragment thereof, wherein the humanized CLDN18.2 antibody includes a light chain variable region and a heavy chain variable region, wherein the heavy chain variable region comprises an amino acid sequence with at least 95%, 96%, 97%, 98%, 99% or 100% sequence identity to the sequence of any one of SEQ ID NOs. 17-21, and/or the light chain variable region comprises an amino acid sequence with at least 95%, 96%, 97%, 98%, 99% or 100% sequence identity to the sequence of any one of SEQ ID NOs. 22-24.

In a further aspect, the antibody or antigen-binding fragment thereof is a humanized antibody or antigen-binding fragment thereof, the humanized CLDN18.2 antibody includes a light chain variable region and a heavy chain variable region, wherein the heavy chain variable region comprises an amino acid sequence with at least 95%, 96%, 97%, 98%, 99% or 100% sequence identity to the sequence of SEQ ID NO. 17, and the light chain variable region comprises an amino acid sequence with at least 95%, 96%, 97%, 98%, 99% or 100% sequence identity to the sequence of SEQ ID NO. 22.

In a further embodiment, the antibody comprises a light chain constant domain, and optionally a heavy chain constant domain.

Optionally, the heavy chain constant domain is a human IgG1 heavy chain constant domain, or the light chain constant domain is a human kappa light chain constant domain. In a further embodiment, the heavy chain constant domain may contain one or more mutations, for example, mutations L234A, L235A and P329A, that reduce or eliminate an effect of ADCC and/or CDC.

In a specific aspect, the present application provides a CD3 antigen binding domain that specifically binds to CD3, wherein the antigen binding fragment comprises a heavy chain variable region and a light chain variable region, wherein
the heavy chain variable region comprises HCDR1 comprising the amino acid sequence represented by SEQ ID NO. 31, HCDR2 comprising the amino acid sequence represented by SEQ ID NO. 32 and HCDR3 comprising the amino acid sequence represented by SEQ ID NO. 33, and/or the light chain variable region comprises LCDR1 comprising the amino acid sequence represented by SEQ ID NO. 34, LCDR2 comprising the amino acid sequence represented by SEQ ID NO. 35 and LCDR3 comprising the amino acid sequence represented by SEQ ID NO. 36;
the heavy chain variable region comprises HCDR1 comprising the amino acid sequence represented by SEQ ID NO. 37, HCDR2 comprising the amino acid sequence represented by SEQ ID NO. 38 and HCDR3 comprising the amino acid sequence represented by SEQ ID NO. 39, and/or the light chain variable region comprises LCDR1 comprising the amino acid sequence represented by SEQ ID NO. 34, LCDR2 comprising the amino acid sequence represented by SEQ ID NO. 35 and LCDR3 comprising the amino acid sequence represented by SEQ ID NO. 36; or
the heavy chain variable region comprises HCDR1 comprising the amino acid sequence represented by SEQ ID NO. 37, HCDR2 comprising the amino acid sequence represented by SEQ ID NO. 40 and HCDR3 comprising the amino acid sequence represented by SEQ ID NO. 39, and/or the light chain variable region comprises LCDR1 comprising the amino acid sequence represented by SEQ ID NO. 34, LCDR2 comprising the amino acid sequence represented by SEQ ID NO. 35 and LCDR3 comprising the amino acid sequence represented by SEQ ID NO. 36.

In a specific aspect, the present application provides a CD3 antigen binding domain that specifically binds to CD3, wherein the antigen binding fragment comprises a heavy chain variable region (VH domain) and a light chain variable region (VL domain) wherein:
the heavy chain variable region comprises an amino acid sequence with at least 95%, 96%, 97%, 98%, 99% or 100% sequence identity to the sequence of SEQ ID NO. 25, and the light chain variable region comprises an amino acid sequence with at least 95%, 96%, 97%, 98%, 99% or 100% sequence identity to the sequence of SEQ ID NO. 26; or
the heavy chain variable region comprises an amino acid sequence with at least 95%, 96%, 97%, 98%, 99% or 100% sequence identity to the sequence of SEQ ID NO. 27, and the light chain variable region comprises an amino acid sequence with at least 95%, 96%, 97%, 98%, 99% or 100% sequence identity to the sequence of SEQ ID NO. 26; or
the heavy chain variable region comprises an amino acid sequence with at least 95%, 96%, 97%, 98%, 99% or 100% sequence identity to the sequence of SEQ ID NO. 28, and the light chain variable region comprises an amino acid sequence with at least 95%, 96%, 97%, 98%, 99% or 100% sequence identity to the sequence of SEQ ID NO. 26.

In a further aspect, the present application provides a bispecific antibody, comprising a first antibody or antigen binding fragment thereof that specifically binds to CLDN18.2 or a naturally occurring variant thereof and a second antigen binding domain that specifically binds to CD3 or a naturally occurring variant thereof.

In a further aspect, in the above-mentioned bispecific antibody, the first antibody or antigen binding fragment thereof is the above-mentioned CLDN18.2 antibody or antigen binding fragment thereof, and the second antigen binding domain is the above-mentioned CD3 antigen binding domain.

In a further aspect, in the above-mentioned bispecific antibody, the first antibody or antigen binding fragment thereof comprises two identical heavy chain and two identical light chains, and the second antigen binding domain comprises two identical single chain antibody fragments (scFv).

In a further aspect, in the above-mentioned bispecific antibody, each of the light chains of the first antibody or antigen binding fragment is fused to each said single chain antibody fragment (scFv) of the second antigen binding domain.

In a further aspect, in the above-mentioned bispecific antibody, the C-terminal of the constant region of each said light chain of the first antibody or antigen binding fragment is fused to the N-terminal of the heavy chain variable region of each said single chain antibody fragment (scFv) of the second antigen binding domain, directly or via a peptide linker.

In a further aspect, the above-mentioned bispecific antibody comprises a monoclonal antibody that is an immunoglobulin that binds to CLDN18.2, said immunoglobulin comprising two identical heavy chains and two identical light chains, wherein said light chains are a first light chain and a second light chain, wherein the first light chain is fused to a first single chain variable fragment (scFv), via a peptide linker, to create a first light chain fusion polypeptide, and wherein the second light chain is fused to a second scFv, via a peptide linker, to create a second light chain fusion polypeptide, wherein the first and second scFv (i) are identical, and (ii) bind to CD3, and wherein the first and second light chain fusion polypeptides are identical.

In one specific aspect, in the above-mentioned bispecific antibody, the C-terminal of the constant region of the first light chain is fused to the N-terminal of the heavy chain variable region of the first scFv directly or via a peptide linker, and the C-terminal of the constant region of the second light chain is fused to the N-terminal of the heavy chain variable region of the second scFv directly or via a peptide linker.

In one specific aspect, in the above-mentioned bispecific antibody, each of the first and second scFv comprises the above mentioned CD3 antigen binding domain.

In a specific aspect, the above-mentioned bispecific antibody provided herein binds to both CLDN18.2 and CD3.

In a specific aspect, the sequence of the above-mentioned peptide linker is shown in SEQ ID NO. 41 or SEQ ID NO. 42.

In a further aspect, the present application provides a bispecific antibody, comprising a first antibody or antigen binding fragment thereof that specifically binds to CLDN18.2 or a naturally occurring variant thereof and a second antigen binding domain that specifically binds to CD3 or a naturally occurring variant thereof, wherein a first antibody or antigen binding fragment thereof includes a light chain variable region and a heavy chain variable region, wherein the heavy chain variable region comprises HCDR1 comprising the amino acid sequence represented by SEQ ID NO. 11, HCDR2 comprising the amino acid sequence represented by SEQ ID NO. 12 and HCDR3 comprising the amino acid sequence represented by SEQ ID NO. 13, and the light chain variable region comprises LCDR1 comprising the amino acid sequence represented by SEQ ID NO. 14, LCDR2 comprising the amino acid sequence represented by SEQ ID NO. 15 and LCDR3 comprising the amino acid sequence represented by SEQ ID NO. 16; and the second antigen binding domain comprises a heavy chain variable region and a light chain variable region, wherein the heavy chain variable region comprises HCDR1 comprising the amino acid sequence represented by SEQ ID NO. 31, HCDR2 comprising the amino acid sequence represented by SEQ ID NO. 32 and HCDR3 comprising the amino acid sequence represented by SEQ ID NO. 33, and the light chain variable region comprises LCDR1 comprising the amino acid sequence represented by SEQ ID NO. 34, LCDR2 comprising the amino acid sequence represented by SEQ ID NO. 35 and LCDR3 comprising the amino acid sequence represented by SEQ ID NO. 36.

In a further aspect, the present application provides a bispecific antibody, comprising a first antibody or antigen binding fragment thereof that specifically binds to CLDN18.2 or a naturally occurring variant thereof and a second antigen binding domain that specifically binds to CD3 or a naturally occurring variant thereof, wherein a first antibody or antigen binding fragment thereof includes a light chain variable region and a heavy chain variable region, wherein the heavy chain variable region comprises HCDR1 comprising the amino acid sequence represented by SEQ ID NO. 11, HCDR2 comprising the amino acid sequence represented by SEQ ID NO. 12 and HCDR3 comprising the amino acid sequence represented by SEQ ID NO. 13, and the light chain variable region comprises LCDR1 comprising the amino acid sequence represented by SEQ ID NO. 14, LCDR2 comprising the amino acid sequence represented by SEQ ID NO. 15 and LCDR3 comprising the amino acid sequence represented by SEQ ID NO. 16; and the second antigen binding domain comprises a heavy chain variable region and a light chain variable region, wherein the heavy chain variable region comprises HCDR1 comprising the amino acid sequence represented by SEQ ID NO. 37, HCDR2 comprising the amino acid sequence represented by SEQ ID NO. 38 and HCDR3 comprising the amino acid sequence represented by SEQ ID NO. 39, and the light chain variable region comprises LCDR1 comprising the amino acid sequence represented by SEQ ID NO. 34, LCDR2 comprising the amino acid sequence represented by SEQ ID NO. 35 and LCDR3 comprising the amino acid sequence represented by SEQ ID NO. 36.

In a further aspect, the present application provides a bispecific antibody, comprising a first antibody or antigen binding fragment thereof that specifically binds to CLDN18.2 or a naturally occurring variant thereof and a second antigen binding domain that specifically binds to CD3 or a naturally occurring variant thereof, wherein a first antibody or antigen binding fragment thereof includes a light chain variable region and a heavy chain variable region, wherein the heavy chain variable region comprises HCDR1 comprising the amino acid sequence represented by SEQ ID NO. 11, HCDR2 comprising the amino acid sequence represented by SEQ ID NO. 12 and HCDR3 comprising the amino acid sequence represented by SEQ ID NO. 13, and the light chain variable region comprises LCDR1 comprising the amino acid sequence represented by SEQ ID NO. 14, LCDR2 comprising the amino acid sequence represented by SEQ ID NO. 15 and LCDR3 comprising the amino acid sequence represented by SEQ ID NO. 16; and the second antigen binding domain comprises a heavy chain variable region and a light chain variable region, wherein the heavy chain variable region comprises HCDR1 comprising the amino acid sequence represented by SEQ ID NO. 37, HCDR2 comprising the amino acid sequence represented by SEQ ID NO. 40 and HCDR3 comprising the amino acid sequence represented by SEQ ID NO. 39, and the light chain variable region comprises LCDR1 comprising the amino acid sequence represented by SEQ ID NO. 34, LCDR2 comprising the amino acid sequence represented by SEQ ID NO. 35 and LCDR3 comprising the amino acid sequence represented by SEQ ID NO. 36.

In a further aspect, the present application provides a bispecific antibody, comprising a first antibody or antigen binding fragment thereof that specifically binds to CLDN18.2 or a naturally occurring variant thereof and a second antigen binding domain that specifically binds to CD3 or a naturally occurring variant thereof, wherein a first antibody or antigen binding fragment thereof includes a light chain variable region and a heavy chain variable region, wherein the heavy chain variable region comprises an amino acid sequence with at least 95%, 96%, 97%, 98%, 99% or 100% sequence identity to the sequence of SEQ ID NO. 17, and the light chain variable region comprises an amino acid sequence with at least 95%, 96%, 97%, 98%, 99% or 100% sequence identity to the sequence of SEQ ID NO. 22; and the second antigen binding domain comprises a heavy chain variable region and a light chain variable region, wherein the heavy chain variable region comprises an amino acid sequence with at least 95%, 96%, 97%, 98%, 99% or 100% sequence identity to the sequence of SEQ ID NO. 25, and the light chain variable region comprises an amino acid sequence with at least 95%, 96%, 97%, 98%, 99% or 100% sequence identity to the sequence of SEQ ID NO. 26.

In a further aspect, the present application provides a bispecific antibody, comprising a first antibody or antigen binding fragment thereof that specifically binds to CLDN18.2 or a naturally occurring variant thereof and a second antigen binding domain that specifically binds to CD3 or a naturally occurring variant thereof, wherein a first antibody or antigen binding fragment thereof includes a light chain variable region and a heavy chain variable region, wherein the heavy chain variable region comprises an amino acid sequence with at least 95%, 96%, 97%, 98%, 99% or 100% sequence identity to the sequence of SEQ ID NO. 17, and the light chain variable region comprises an amino acid sequence with at least 95%, 96%, 97%, 98%, 99% or 100% sequence identity to the sequence of SEQ ID NO. 22; and the second antigen binding domain comprises a heavy chain variable region and a light chain variable region, wherein the heavy chain variable region comprises an amino acid sequence with at least 95%, 96%, 97%, 98%, 99% or 100% sequence identity to the sequence of SEQ ID NO. 27, and the light chain variable region comprises an amino acid sequence with at least 95%, 96%, 97%, 98%, 99% or 100% sequence identity to the sequence of SEQ ID NO. 26.

In a further aspect, the present application provides a bispecific antibody, comprising a first antibody or antigen binding fragment thereof that specifically binds to CLDN18.2 or a naturally occurring variant thereof and a second antigen binding domain that specifically binds to CD3 or a naturally occurring variant thereof, wherein a first antibody or antigen binding fragment thereof includes a light chain variable region and a heavy chain variable region, wherein the heavy chain variable region comprises an amino acid sequence with at least 95%, 96%, 97%, 98%, 99% or 100% sequence identity to the sequence of SEQ ID NO. 17, and the light chain variable region comprises an amino acid sequence with at least 95%, 96%, 97%, 98%, 99% or 100% sequence identity to the sequence of SEQ ID NO. 22; and the second antigen binding domain comprises a heavy chain variable region and a light chain variable region, wherein the heavy chain variable region comprises an amino acid sequence with at least 95%, 96%, 97%, 98%, 99% or 100% sequence identity to the sequence of SEQ ID NO. 28, and the light chain variable region comprises an amino acid sequence with at least 95%, 96%, 97%, 98%, 99% or 100% sequence identity to the sequence of SEQ ID NO. 26.

In a further aspect, the above-mentioned bispecific antibodies provided herein have advantageous properties such as high producibility, stability, binding affinity, biological activity, specific targeting of certain T cells, targeting efficiency, remaining tumor cell killing and reduced toxicity.

In a further aspect, the above-mentioned bispecific antibody provided here shows reduced binding affinity with CD3 compared to wild-type CD3 binders.

In a further aspect, the above-mentioned bispecific antibodies provided herein show reduced TCR signaling strength.

In a further aspect, the above-mentioned bispecific antibodies provided herein show reduced cytokine release mediated by mutant CD3 binder variants.

In a further aspect, the present application also provides an isolated nucleic acid comprising nucleic acid sequences encoding the above-described CLDN18.2 antibody or antigen binding fragment thereof or the above-mentioned bispecific antibody.

In a further aspect, the present application also provides a vector comprising the nucleic acid encoding the above-mentioned CLDN18.2 antibody or antigen binding fragments thereof or the above-mentioned bispecific antibody.

In a further aspect, the disclosure also provides an isolated host cell comprising the above-mentioned vector or the above-mentioned isolated nucleic acid.

The appropriate host cells are transformed with the above-mentioned vector, and the above-mentioned host cells expressing the above-mentioned CLDN18.2 antibody or antigen binding fragments thereof or the above-mentioned bispecific antibody are obtained.

In a further aspect, the disclosure also provides various known host cell/expression vector combinations for antibody preparation by introducing isolated antibody genes into appropriate hosts. Appropriate eukaryotic cells used as host cells include animal cells, plant cells, and fungal cells. Specifically, the animal cells include, for example, the following cells: (1) mammalian cells: CHO, COS, myeloma, baby hamster kidney (BHK), HeLa, Vero, or such; (2) amphibian cells: Xenopus oocytes, or such; and (3) insect cells: sf9, sf21, Tn5, or such.

In a further aspect, the disclosure also provides a method of producing the above-mentioned antibody or antigen binding fragment thereof or the above-mentioned bispecific antibody, comprising culturing the above-mentioned host cell so that the antibody or antigen binding fragment thereof or the bispecific antibody is produced. In a further aspect, the method further comprises recovering the antibody or antigen binding fragment thereof or the bispecific antibody produced by the host cell.

In a further aspect, the disclosure also provides the isolated antibody or antigen binding fragment thereof or the bispecific antibody produced by the above-mentioned method.

In a further aspect, the disclosure also provides a pharmaceutical composition comprising the above-mentioned CLDN18.2 antibody or antigen binding fragment thereof or the above-mentioned bispecific antibody, nucleic acid, vector, the host cell and optionally a pharmaceutically acceptable carrier.

In a further aspect, the disclosure also provides a method of treating cancer in a subject in need thereof, comprising administering to the subject the above-mentioned pharmaceutical composition comprising the above-mentioned CLDN18.2 antibody or antigen binding fragment thereof or the above-mentioned bispecific antibody, nucleic acid, vector, the host cell and optionally a pharmaceutically acceptable carrier.

In a further aspect, the above-mentioned cancer is a CLDN18.2-positive cancer.

In a further aspect, the cancer is hematological cancer or solid cancer.

In a further aspect, the CLDN18.2-positive cancer includes but is not limited to gastric cancer or pancreatic cancer.

The technical solutions of the present application have one or more of the following advantages:
1. The CLDN18.2 antibody or antigen binding fragments are highly selective and specific to CLDN18.2; the antibodies have particularly advantageous properties such as high producibility, stability, binding affinity, biological activity, specific targeting of CLDN18.2-positive cells, efficient TCR signaling strength, targeting efficiency, remaining tumor cell killing and reduced cytotoxicity.
2. The above-mentioned bispecific antibodies provided herein have advantageous properties such as high producibility, stability, binding affinity, biological activity, specific targeting of certain T cells, targeting efficiency, remaining tumor cell killing and reduced toxicity.
3. The above-mentioned bispecific antibody with mutant CD3 binders provided herein shows reduced binding affinity with CD3, and reduced cytokines release mediated by mutant CD3 binder variants compared to bispecific antibody with wild-type CD3 binders.
4. The application enables desirable treatment for cancer which has not only a high level of safety but also reduced physical burden, and is highly convenient for patients.

### BRIEF DESCRIPTION OF THE DRAWINGS

The novel features of the present application are set forth with particularity in the appended claims. Some of the features and advantages of the present application are explained in the following detailed description in the embodiments and in the examples.

FIG. 1 shows an illustration of CLDN18.2/CD3 bispecific antibody structure. Each anti-CD3 single-chain variable fragment (scFv) is linked to the C-terminus of an anti-CLDN18.2 light chain. The anti-CD3 sequence contains key point mutations (L234A, L235A and P329A) that abrogate binding of Fcγ receptors (FcγR), abolishing antibody directed cytotoxicity (ADCC) and complement-dependent cytotoxicity (CDC) effector functions.

FIG. 2A-2C. Confirmation of binding affinity of CLDN18.2 monoclonal antibody m-13 to hCLDN18.2 protein (FIG. 2A), CLDN18.2 overexpressing HEK293 cells (FIG. 2B) and SNU601 cells (FIG. 2C).

FIG. 3A-3B. Comparison of binding affinity of CLDN18.2/CD3 bispecific antibodies (h-808/CD3-p, h-841/CD3-p, m-13/CD3-p, m-6/CD3-p) with Amgen CLDN18.2/CD3 bispecific antibody(AMG-910). CLDN18.2 overexpressing HEK293 cells (FIG. 3A) and CD3 positive Jurkat cells (FIG. 3B) were stained with CLDN18.2/CD3 bispecific antibodies in serial dilution of concentrations. Mean fluorescence intensity (MFI) was determined by flow cytometry.

FIG. 4. TCR-NFAT activity mediated by CLDN18.2/CD3 bispecific antibodies in NFAT-reporter Jurkat cells. NFAT reporter Jurkat cells were cocultured with CLDN18.2-expressing SNU620 cells (E/T: 3/1) in serial dilutions of CLDN18.2/CD3 bispecific antibodies for 6 hours. NFAT activity was determined by relative luminescence units (RLU).

FIG. 5A-5F. Comparison of killing activity mediated by murine CLDN18.2/CD3 bispecific antibodies against target cells with various expression levels of CLDN18.2. CLDN18.2 overexpressing HEK (i.e. CLDN18.2⁺HEK or CLDN18.2 overexpressing HEK293) (FIG. 5A), medium CLDN18.2 expressing SNU620 (FIG. 5B) and low CLDN18.2 expressing KATO III (FIG. 5C) target cells were harvested, counted and seeded in 96-well plates. Effector PBMCs were added to each well in a proportion of 20:1 in RPMI 1640. Serial dilutions of the indicated antibodies were performed in RPMI 1640 medium and added to the target/effector-containing well. The plates were incubated for 48 hours, and Luciferase intensity was determined. FIG. 5D-5F shows EC50 of killing potency for murine CLDN18.2/CD3 bispecific antibodies.

FIG. 6. Comparison of cytokine release levels mediated by murine CLDN18.2/CD3 bispecific antibodies. Freshly isolated PMBCs were isolated from healthy donors and cocultured with CLDN18.2-expressing HEK (i.e. CLDN18.2⁺HEK), SNU620, and KATO III target cells in serial dilution of CLDN18.2/CD3 bispecific antibodies. The supernatants were collected at 48 hours. IFN-γ and TNF-α were determined by ELISA assays.

FIG. 7. Like Amgen CLDN18.2 antibodies (i.e. AMG-910), the murine CLDN18.2 antibodies (i.e. m-6/CD3-p, m-12/CD3-p, m-13/CD3-p) and h841/CD3-p antibody showed no cross-reactivity with CLDN18.1. CLDN18.1-overexpressing CHO cells were stained with h808/CD3, h841/CD3, three murine CLDN18.2/CD3 or Amgen CLDN18.2/CD3 bispecific antibodies. The top panel shows staining CLDN18.1-DYK Tag expressing CHO with DYKDDDK Epitope Tag antibody, anti-human IgG control, h808/CD3, and h841/CD3 bispecific antibodies. The bottom panel shows staining CLDN18.1-DYK tag expressing CHO cells with m-6, m-12, m-13, and Amgen CLDN18.2/CD3 bispecific antibodies.

FIG. 8. Freshly isolated T cells (Effector) were cocultured with CLDN18.2 expressing SNU620 Luc target cells (T) at E/T ratio:20/1 in a serial dilution of CLDN18.2/CD3 bispecific antibodies (CLDN18.2-808/CD3 and CLDN18.2-841/CD3 bispecific antibodies) for 48 hours. Luciferase quantitative assays were performed to determine cytotoxic activity against target cells.

FIG. 9. Murine CLDN18.2-12 clone nonspecifically binds to CLDN18.2 negative LoVo and LS-174T cells. CLDN18.2 negative LoVo tumor cells (the top panel) and CLDN18.2 negative LS-174T tumor cells (the bottom panel) were stained with m-6, m-12, and m-13/CD3 bispecific antibodies.

FIG. 10. Humanized CLDN18.2-13 subclones show similar binding affinity to CLDN18.2 on the cell surface. CLDN18.2-13 subclones VH1-5+VL1, parental m-13 and Amgen CLDN18.2/CD3 bispecific antibodies were used to stain CLDN18.2 overexpressing HEK cells (the top left panel); h-13 subclones VH1-5+VL2, parental m-13 and Amgen CLDN18.2/CD3 bispecific antibodies were used to stain CLDN18.2 natural expressing SNU620 cells (the top right panel); h-13 subclones VH1-5+VL3, m-13 parental and Amgen CLDN18.2/CD3 bispecific antibodies were used to stain CLDN18.2 overexpressing HEK cells (the left of the bottom panel); Humanness score of murine CLDN18.2-13 was shown in the bottom right panel.

FIG. 11. High specificity of CLDN18.2-13/CD3 bispecific antibodies. CLDN18.1-overexpressing CHO cells were stained with h-13 VH1-5+VL1 (the top panel), VH1-5+VL2 (the middle panel), and LV3+HV1-5 (the bottom panel) subclone/CD3 bispecific antibodies.

FIG. 12. Comparable NFAT activity mediated by humanized CLDN18.2-13/CD3-p bispecific antibodies as the parental murine CLDN18.2-13/CD3-p. NFAT reporter Jurkat cells were cocultured with CLDN18.2 expressing SNU620 cells (E/T: 3/1) in serial dilutions with hCLDN18.2-13 VL1+VH1-5 (the left panel), VL2+VH1-5 (the middle panel), and VL3+VH1-5 (the right panel) subclone/CD3 bispecific antibodies for 6 hours. NFAT activity was determined by relative luminescence units (RLU).

FIG. 13. Comparable killing activity mediated by humanized CLDN18.2/CD3 and murine CLDN18.2-13/CD3 bispecific antibodies. CLDN18.2-expressing SNU620 and CLDN18.2-overexpressing HEK cells were harvested, counted and seeded in 96-well plates. Effector PBMCs were added to each well in a proportion of 20:1 in serial dilutions of VL1+VH1-5 (the top left panel), VL1+VH1-5 (the top right panel), VL3+HV1-5 (the bottom left panel), and VL3+VL1-5 (the bottom right panel) bispecific antibodies. The plates were incubated for 48 hours, and Luciferase intensity was determined.

FIG. 14A-14D. Comparison of binding affinity of humanized CLDN18.2/CD3 bispecific antibodies with murine parental CLDN18.2-13/CD3 bispecific antibody. CLDN18.2-expressing SNU620 cells (FIG. 14A) and Jurkat cells (FIG. 14C) were stained with h-13/CD3, m-13/CD3, Amgen CLDN18.2/CD3 bispecific antibodies or IgG control, followed by a PE-conjugated anti-human IgG ab. Mean fluorescence intensity (MFI) was determined by flow cytometry. FIG. 14B and FIG. 14D shows EC50 of binding affinity of humanized CLDN18.2/CD3 bispecific antibodies with murine parental CLDN18.2-13/CD3 bispecific antibody.

FIG. 15. Comparison of TCR-NFAT signaling mediated by CLDN18.2-13/CD3 bispecific antibodies. NFAT reporter Jurkat cells were cocultured with CLDN18.2-expressing SNU620 cells (E/T: 3/1) in serial dilutions of h-13/CD3, m-13/CD3, AMG-910 for 6 hours. NFAT activity was determined by relative luminescence units (RLU).

FIG. 16. Comparison of killing potency mediated by h-13/CD3 bispecific antibodies against various CLDN18.2-expressing targets. CLDN18.2-overexpressing HEK (i.e. CLDN18.2 HEK), CLDN18.2 naturally expressing SNU620 and CLDN18.2 naturally expressing KATO III target cells were harvested, counted and seeded in 96-well plates. Effector PBMCs were added to each well in a proportion of 20:1 in RPMI 1640. Serial dilutions of the indicated antibodies were performed in RPMI 1640 medium and added to the target/effector-containing well. The plates were incubated for 48 hours, and Luciferase intensity was determined.

FIG. 17. Comparison of cytokine release mediated by CLDN18.2-13/CD3 bispecific antibodies. Freshly isolated PBMCs were isolated from healthy donors and cocultured with CLDN18.2-expressing SNU620 target cells in serial dilutions of the indicated CLDN18.2/CD3 bispecific antibodies with various binding affinities of CD3. The supernatants were collected at 48 hours. IFN-γ, TNF-α, IL-2, and IL-10 concentrations were determined by ELISA assays.

### DETAILED DESCRIPTION OF THE INVENTION

Unless defined otherwise, technical and scientific terms used herein have the same meaning as generally used in the art to which this disclosure belongs. For purposes of interpreting this specification, the following definitions will apply and whenever appropriate, terms used in the singular will also include the plural and vice versa. As used herein and in the appended claims, the singular forms “a”, “an”, and “the” also refer to the plural forms unless the context clearly dictates otherwise, e.g., reference to “a host cell” includes a plurality of such host cells.

As used herein, the term “antigen binding fragment” or “antigen binding molecule” refers in its broadest sense to a molecule that specifically binds an antigenic determinant. Examples of antigen binding molecules are antibodies, antibody fragments and scaffold antigen binding proteins. The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

A humanized antibody is also called a reshaped human antibody. Specifically, humanized antibodies prepared by grafting the CDR of a non-human animal antibody such as a mouse antibody to a human antibody are known in the art. Common genetic engineering techniques for obtaining humanized antibodies are also known. Specifically, for example, overlap extension PCR is known as a method for grafting a mouse antibody CDR to a human FR. In overlap extension PCR, a nucleotide sequence encoding a mouse antibody CDR to be grafted is added to primers for synthesizing a human antibody FR. Primers are prepared for each of the four FRs. It is generally considered that when grafting a mouse CDR to a human FR, selecting a human FR that has high identity to a mouse FR is advantageous for maintaining the CDR function. That is, it is generally preferable to use a human FR comprising an amino acid sequence which has high identity to the amino acid sequence of the FR adjacent to the mouse CDR to be grafted.

The term “bispecific” means that the antibody is able to specifically bind to at least two distinct antigenic determinants, for example two binding sites each formed by a pair of an antibody heavy chain variable domain (VH) and an antibody light chain variable domain (VL) binding to different antigens or to different epitopes on the same antigen. Such a bispecific antibody is referred to as a 1+1 format. Other bispecific antibody formats are 2+1 formats (comprising two binding sites for a first antigen or epitope and one binding site for a second antigen or epitope) or 2+2 formats (comprising two binding sites for a first antigen or epitope and two binding sites for a second antigen or epitope). Typically, a bispecific antibody comprises two antigen binding sites, each of which is specific for a different antigenic determinant. The term “valent” as used within the current application denotes the presence of a specified number of binding domains in an antigen binding molecule. As such, the terms “bivalent”, “tetravalent”, and “hexavalent” denote the presence of two binding domains, four binding domains, and six binding domains, respectively, in an antigen binding molecule. The bispecific antibodies according to the disclosure are at least “bivalent” and may be “trivalent” or “multivalent” (e.g., “tetravalent” or “hexavalent”). In a particular aspect, the antibodies of the present disclosure have two or more binding sites and are bispecific. That is, the antibodies may be bispecific even in cases where there are more than two binding sites (i.e. that the antibody is trivalent or multivalent). The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are hetero-tetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g., γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies, triabodies, tetrabodies, cross-Fab fragments; linear antibodies; single-chain antibody molecules (e.g., scFv); multispecific antibodies formed from antibody fragments and single domain antibodies. Single-domain antibodies are antibody fragments comprising all or a portion of the heavy chain variable domain or all or a portion of the light chain variable domain of an antibody. In certain embodiments, a single-domain antibody is a human single-domain antibody. In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full-length antibodies. Antibody fragments can be made by various techniques, including but not limited to proteolytic digestion of an intact antibody as well as production by recombinant host cells (e.g., E. coli or phage), as described herein. Papain digestion of intact antibodies produces two identical antigen-binding fragments, called “Fab” fragments, each containing the heavy- and light-chain variable domains and also the constant domain of the light chain and the first constant domain (CH1) of the heavy chain. As used herein, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteines from the antibody hinge region. Fab′-SH are Fab′ fragments wherein the cysteine residue(s) of the constant domains bear a free thiol group. Pepsin treatment yields an F(ab′)2 fragment that has two antigen-combining sites (two Fab fragments) and a part of the Fc region.

A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. In addition, antibody fragments comprising single chain polypeptides have the characteristics of a VH domain, namely being able to assemble together with a VL domain, or of a VL domain, namely being able to assemble together with a VH domain to a functional antigen binding site, thereby providing the antigen binding properties of full-length antibodies.

By “specific binding” it is meant that the binding is selective for the antigen and can be distinguished from unwanted or non-specific interactions with substrates other than the antigen. The ability of an antigen binding molecule to bind to a specific antigen can be measured either through an enzyme-linked immunosorbent assay (ELISA) or other techniques familiar to one of skill in the art, and traditional binding assays. In one embodiment of the present application, the extent of binding of an antigen binding molecule to an unrelated protein is less than about 10% of the binding of the antigen binding molecule to the antigen as measured, e.g., by SPR. In certain embodiments, a molecule that binds to the antigen has a dissociation constant (Kd) of <1 μM, <100 nM, <10 nM, <1 nM, <0.1 nM, <0.01 nM, or <0.001 nM (e.g., 10-7 M or less, e.g., from 10-7 M to 10-13 M, e.g., from 10-9 M to 10-13 M).

“Affinity” or “binding affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR). As used herein, the term “high affinity” of an antibody refers to an antibody having a Kd of 10⁻⁹ M or less and even more particularly 10⁻¹⁰ M or less for a target antigen. The term “low affinity” of an antibody refers to an antibody having a Kd of 10⁻⁸ or higher.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). A single VH or VL domain may be sufficient to confer antigen-binding specificity.

Hypervariable regions (HVRs) are also referred to as complementarity determining regions (CDRs), and these terms are used herein interchangeably in reference to portions of the variable region that form the antigen binding regions. This particular region has been described by Kabat et al., U.S. Dept. of Health and Human Services, where the definitions include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services. Unless otherwise specified, references to the numbering of specific amino acid residue positions in an antibody variable region herein are made according to the Kabat numbering system. With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. Unless otherwise indicated, HVR residues and other residues in the variable domain (e.g., FR residues) are numbered herein according to Kabat et al.

By “fused to” or “connected to”, it is meant that the components (e.g., an antigen binding domain and a FC domain) are linked by peptide bonds, either directly or via one or more peptide linkers.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages.

A “therapeutically effective amount” of an agent, e.g., a pharmaceutical composition, refers to an amount effective, at dosages and for periods of time necessary, to achieve the desired therapeutic or prophylactic result. A therapeutically effective amount of an agent, for example, eliminates, decreases, delays, minimizes or prevents adverse effects of a disease.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Particularly, the individual or subject is a human. The term “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the present application are used to delay development of a disease or to slow the progression of a disease.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewing's sarcoma, including refractory versions of any of the above cancers, or a combination of one or more of the above cancers.

### Definitions

For purposes of interpreting the antibodies used in the following examples, the following definitions are provided.
1. Definition of anti-CLDN18.2 monoclonal antibodies used in the following examples:
1) 2 lead humanized CLDN18.2 antibodies: hCLDN18.2 841 (i.e. h841), hCLDN18.2 808 (i.e. h808), and sequences thereof are shown in Table 1;
2) 3 lead murine anti-human CLDN18.2 antibodies: m-anti-human CLDN18.2-6 (i.e. m-6), m-anti-human CLDN18.2-12 (i.e. m-12), and m-anti-human CLDN18.2-13 (i.e. m-13), and sequences thereof are shown in Table 1;
3) humanized m-13: h-13, and VH and VL thereof are shown in Table 2;
4) humanized m-6: h-6, and VH and VL thereof are shown in Table 2-1.
2. Definition of anti-CD3 antibody used in the following examples:
1) humanized anti-CD3 antibody: SP34(i.e. CD3-p or CD3 Parental, mutant CD3 antibody (CD3-v1, CD3-v2); and the sequences thereof are shown in Table 3;
3. Definition of anti-CLDN18.2/CD3 BsAbs used in the following examples:
1) CLDN18.2/CD3 bsAbs:
\textcircled{1}h808/CD3-p, h841/CD3-p, m-6/CD3-p, m-13/CD3-p;
\textcircled{2}h841/CD3-v1, h841/CD3-v2;
2) CLDN18.2/CD3 bsAbs using humanized m-13, that is, CLDN18.2-13/CD3 bispecific antibodies or h-13/CD3 bsAbs:
\textcircled{1}CLDN18.2-13/CD3 bispecific antibodies (i.e. h-13/CD3 bsAbs) with CD3-p (i.e. CD3 Parental) arm (i.e. 15 humanized CLDN18.2-13/CD3 bispecific antibodies):
h-13VH1-VL1/CD3-p, h-13VH2-VL1/CD3-p, h-13VH3-VL1/CD3-p, h-13VH4-VL1/CD3-p, h-13VH5-VL1/CD3-p,
h-13VH1-VL2/CD3-p, h-13VH2-VL2/CD3-p, h-13VH3-VL2/CD3-p, h-13VH4-VL2/CD3-p, h-13VH5-VL2/CD3-p,
h-13VH1-VL3/CD3-p, h-13VH2-VL3/CD3-p, h-13VH3-VL3/CD3-p, h-13VH4-VL3/CD3-p, h-13VH5-VL3/CD3-p,
\textcircled{2}CLDN18.2-13/CD3 bispecific antibodies (i.e. h-13/CD3 bsAbs) with mutant CD3 arm (CD3-v1 or CD3-v2):
h-13VH1-VL1/CD3-v1, h-13VH1-VL1/CD3-v2, h-13VH1-VL2/CD3-v2, and hVH1-VL3/CD3-v2
3) Benchmark bispecific antibody: AMG-910.

TABLE 1
The VH or VL sequence of the anti-CLDN18.2 monoclonal antibodies
(Underlined Sequences represent CDRs, the analysis
system is Kabat system)

| Clones | Heavy Chain (VH) | Light Chain (VL) |
|---|---|---|
| hCLDN18.2 | QVQLVQSGAEVKKPGSSVKVSCK | DIQMTQSPSTLSASVGDRVTITCRA |
| 841 | ASGGTFSSYAISWVRQAPGQGLEW | SQSINSWLAWYQQKPGKAPKLLIY |
| | MGRIIPILGIANYAQKFQGRVTVTA | KASSLESGVPSRFSGSGSGTEFTLTI |
| | DKSTSTAYMELSSLRSEDTAVYYC | SSLQPDDFATYYCQQYNSFPLTFGG |
| | ARERDNWDPYYYYYGMDVWGQ | GTKVEIK (SEQ ID NO. 2) |
| | GTTVTVSS (SEQ ID NO. 1) | |
| hCLDN18.2 | EVQLVQSGGGVVQPGRSLRLSCA | DIVMTQSPSSLSASVGDRVTITCRA |
| 808 | ASGFTFSSYAMHWVRQAPGKGLE | SQGIRNDLGWYQQKPGKAPKLLIYA |
| | WVAVISYDGSNKYYADSVKGRFTI | ASSLQSGVPSRFSGSGSGTDFTLTI |
| | SRDNSKNTLYLQMNSLRAEDTAV | SSLQPEDFATYYCLQDYNYPRTFGQ |
| | YYCARELRFFDWLLGSAFDIWGQ | GTKVEIK (SEQ ID NO. 4) |
| | GTTVTVSS (SEQ ID NO. 3) | |
| m-6 | EVQLVESGGGLVQPGGSLKLSCAA | DIQITQSPSSLTVTAGEKVTMSCQS |
| | SGFTFSSYGMSWVRQTPDKRLELV | SQSLLNSGNHRNYLTWYQQKPGQPP |
| | ATINSNGGSTYYPDSVKGRFTISR | KLLMYWASTRESGVPDRFAGSGSGT |
| | DNAKNTLYLQMSSLKSEDTAMYYC | DFTLTISSVQAEDLAIYYCQNTYYY |
| | ARLSYGNSFANWGQGTLVTVSS | PFTFGSGTKLEIK |
| | (SEQ ID NO. 5) | (SEQ ID NO. 6) |
| m-12 | DVQLQQSGPELMKPGASVKISCKA | DIVMTQSPSSLTVTAGEKVTMNCK |
| | SDYSFTSYNMHWVKQSHGKSLEW | SSQSLLNSGNQKNYLTWYQQKPGQ |
| | IGYIDPFNGGTRYNQKFKGKATLT | PPKLLNYWASTRESGVPDRFTGSG |
| | VDKSSSTAYMHISSLTSEDSAVYY | SGTDFTLTIGSVQAEDLAVYYCQN |
| | CARGAYYGNSFDYWGQGTLVTVS | AYSFPFTFGSGTKLEIK |
| | S (SEQ ID NO. 7) | (SEQ ID NO. 8) |
| m-13 | QLQLQQSGTELVRPGSSVKISCKA | DIVLTQSPSSLTVTAGEKVTMSCK |
| | SGYAFSNYWMNWVKQRPGQGLEW | SSQSLLNSGNQKNYLTWYQQKPGQ |
| | IGQIYPGNGDTKYNGNFKGKATLT | PPKLLIYWASTRDSGVPARFTGSG |
| | ADKSSSTAYMQLSSLTSEDSADYF | SGTDFTLTINSVQAEDLAVYYCQN |
| | CARYYRGNCFDYWGQGTTLTVSS | AYTYPLTFGAGTKLEIK |
| | (SEQ ID NO. 9) | (SEQ ID NO. 10) |
| CDRs of | GYAFSNYWMN (SEQ ID NO. 11) | KSSQSLLNSGNQKNYLT |
| m-13 | QIYPGNGDTKYNGNFKG | (SEQ ID NO. 14) |
| | (SEQ ID NO. 12) | WASTRDS (SEQ ID NO. 15) |
| | YYRGNCFDY(SEQ ID NO. 13) | QNAYTYPLT (SEQ ID NO. 16) |

TABLE 2
Sequences of humanized m-13(i.e. h-13) and VH
and VL thereof (Underlined Sequences represent
CDRs, the analysis system is Kabat system)

| | |
|---|---|
| VH1 | QVQLVQSGAEVKKPGASVKVSCKASGYAFSNYWMNWVRQAPGQRLEWMGQIYP |
| | GNGDTKYNGNFKGRVTITRDTSASTAYMELSSLRSEDTAVYYCARYYRGNCFDY |
| | WGQGTLVTVSS(SEQ ID NO. 17) |
| VH2 | QVQLVQSGAEVKKPGASVKVSCKASGYAFSNYWMNWVRQAPGQGLEWIGQIYP |
| | GNGDTKYNGNFKGRVTITADTSASTAYMELSSLRSEDTAVYFCARYYRGNCFD |
| | YWGQGTLVTVSS(SEQ ID NO. 18) |
| VH3 | QVQLVQSGAEVKKPGASVKVSCKASGYAFSNYWMNWVKQAPGQGLEWIGQIYP |
| | GNGDTKYNGNFKGRVTITADTSASTAYMELSSLRSEDTAVYFCARYYRGNCFD |
| | YWGQGTLVTVSS(SEQ ID NO. 19) |
| VH4 | QVQLVQSGAEVKKPGASVKVSCKASGYAFSNYWMNWVKQAPGQGLEWIGQIYP |
| | GNGDTKYNGNFKGRVTLTADTSASTAYMELSSLRSEDTAVYFCARYYRGNCFDY |
| | WGQGTLVTVSS(SEQ ID NO. 20) |
| VH5 | QVQLVQSGAEVKKPGASVKVSCKASGYAFSNYWMNWVKQAPGQGLEWIGQIYP |
| | GNGDTKYNGNEKGRATLTADTSASTAYMELSSLRSEDTA VYFCARYYRGNCF |
| | DYWGQGTLVTVSS(SEQ ID NO. 21) |
| VL1 | DIVMTQSPDSLAVSLGERATINCKSSQSLLNSGNQKNYLTWYQQKPGQPPKLLIYW |
| | ASTRDSGVPDRFSGSGSGTDFTLTISSLQAEDVAVYYCQNAYTYPLTFGGGTKVEI |
| | K(SEQ ID NO. 22) |
| VL2 | DIVMTQSPDSLAVSLGERATINCKSSQSLLNSGNQKNYLTWYQQKPGQPPKLLIYW |
| | ASTRDSGVPDRFSGSGSGTDFTLTISSVQAEDVAVYYCQNAYTYPLTFGGGTKVEI |
| | K(SEQ ID NO. 23) |
| VL3 | DIVMTQSPDSLAVSLGERATMNCKSSQSLLNSGNQKNYLTWYQQKPGQPPKLLIY |
| | WASTRDSGVPDRFSGSGSGTDFTLTISSVQAEDVAVYYCQNAYTYPLTFGGGTKV |
| | EIK(SEQ ID NO. 24) |

TABLE 2-1
Sequences of humanized m-6(i.e. h-6)
and VH and VL thereof

| | | |
|---|---|---|
| Clones | Heavy Chain (VH) | Light Chain (VL) |
| h-6 | EVQLVESGGGLVQPGGSLRL | DIVITQSPSSLSVTLGERAT |
| | SCAASGFTFSSYGMSWVRQA | LSCRSSQSLLNSGNHRNYLT |
| | PGKGLEWVATINSNGGSTYY | WYQQKPGQAPRLLMYWASTR |
| | PDSVKGRFTISRDNAKNTLY | ESGVPDRFSGSGSGTDFTLT |
| | LQMNSLRAEDTAVYYCARLS | ISSLQPEDFATYYCQNTYYY |
| | YGNSFANWGQGTTVTVSS | PFTFGGGTKLEIK |
| | (SEQ ID NO. 29) | (SEQ ID NO. 30) |

TABLE 3
VH and VL sequences of CD3 arm (CD3 Parental and CD3-v1, CD3-v2)
(Underlined Sequences represent CDRs, the analysis
system is Kabat system)

| Clones | Heavy Chain (VH) | Light Chain (VL) |
|---|---|---|
| CD3 | EVQLVESGGGLVQPGGSLKLSCAA | QTVVTQEPSLTVSPGGTVTLTCGSS |
| Parental | SGFTFNKYAMNWVRQAPGKGLEW | TGAVTSGYYPNWVQQKPGQAPRGL |
| (i.e. | VARIRSKYNNYATYYADSVKDR | IGGTKFLAPGTPARFSGSLLGGKA |
| CD3-p) | FTISRDDSKNTAYLQMNNLKTEDT | ALTLSGVQPEDEAEYYCALWYSNR |
| | AVYYCVRHGNFGNSYISYWAYW | WVFGGGTKLTVL |
| | GQGTLVTVSS | (SEQ ID NO. 26) |
| | (SEQ ID NO. 25) | |
| CD3-V1 | EVQLVESGGGLVKPGGSLKLSCAA | QTVVTQEPSLTVSPGGTVTLTCGSS |
| | SGFTFSTYAMNWVRQAPGKGLEW | TGAVTSGYYPNWVQQKPGQAPRGL |
| | VARIRSKYNNYATYYADSVKDRF | IGGTKFLAPGTPARFSGSLLGGKA |
| | TISRDDSKNTAYLQMNNLRTEDT | ALTLSGVQPEDEAEYYCALWYSNR |
| | AVYYCVRHGNWGNSYISYWAYWG | WVFGGGTKLTVL |
| | QGTTVTVSS (SEQ ID NO. 27) | (SEQ ID NO. 26) |
| CD3-V2 | EVQLVESGGGLVKPGGSLKLSCAA | QTVVTQEPSLTVSPGGTVTLTCGSS |
| | SGFTFSTYAMNWVRQAPGKGLEWV | TGAVTSGYYPNWVQQKPGQAPRGL |
| | ARIRSKYNNYATTYADSVKDRFTI | IGGTKFLAPGTPARFSGSLLGGKA |
| | SRDDSKNTAYLQMNNLRTEDTAVY | ALTLSGVQPEDEAEYYCALWYSNR |
| | YCVRHGNWGNSYISYWAYWGQG | WVFGGGTKLTVL |
| | TTVTVSS (SEQ ID NO. 28) | (SEQ ID NO. 26) |
| CDRs of | SEQ ID NO. 31: KYAMN | SEQ ID NO. 34: GSSTGAVTSGYYPN |
| CD3 | SEQ ID NO. 32: | SEQ ID NO. 35: GTKFLAP |
| Parental | RIRSKYNNYATYYADSVKD | SEQ ID NO. 36: ALWYSNRWV |
| | SEQ ID NO. 33: | |
| | HGNFGNSYISYWAY | |
| CDRs of | SEQ ID NO. 37: TYAMN | SEQ ID NO. 34: GSSTGAVTSGYYPN |
| CD3-V1 | SEQ ID NO. 38: | SEQ ID NO. 35: GTKFLAP |
| | RIRSKYNNYATYYADSVKD | SEQ ID NO. 36: ALWYSNRWV |
| | SEQ ID NO. 39: | |
| | HGNWGNSYISYWAY | |
| CDRs of | SEQ ID NO. 37: TYAMN | SEQ ID NO. 34: GSSTGAVTSGYYPN |
| CD3-V2 | SEQ ID NO. 40: | SEQ ID NO. 35: GTKFLAP |
| | RIRSKYNNYATTYADSVKD | SEQ ID NO. 36: ALWYSNRWV |
| | SEQ ID NO. 39: | |
| | HGNWGNSYISYWAY | |

TABLE 4
Linker and constant region used in the anti-
CLDN18.2 clones or CLDN18.2-13/CD3
bispecific antibodies

| | | SEQ |
|---|---|---|
| DESCRIPTION | SEQUENCE | ID NO: |
| Linker-1 | GGGGSGGGGSGGGGS | 41 |
| Linker-2 | GGGGSGGGGSGGGGSGGGGSGGGGSGGGGS | 42 |
| CL (constant region of | RTVAAPSVFIFPPSDEQLKSGTASVVCLLNNFYP | 43 |
| human kappa light chain) | REAKVQWKVDNALQSGNSQESVTEQDSKDSTYSL | |
| | SSTLTLSKADYEKHKVYACEVTHQGLSSPVTKSF | |
| | NRGECTS | |
| CH1 of human IgGI heavy | ASTKGPSVFPLAPSSKSTSGGTAALGCLVKDYFPE | 44 |
| chain | PVTVSWNSGALTSGVHTFPAVLQSSGLYSLSSVV | |
| | TVPSSSLGTQTYICNVNHKPSNTKVDKKV | |
| Hinge | EPKSCDKTHTCPPCP | 45 |
| CH2 of human IgG1 heavy | APEAAGGPSVFLFPPKPKDTLMISRTPEVTCVVV | 46 |
| chain | DVSHEDPEVKFNWYVDGVEVHNAKTKPREEQYNS | |
| | TYRVVSVLTVLHQDWLNGKEYKCKVSNKALAA | |
| | PIEKTISKAK | |
| CH3 of human IgG1 heavy | GQPREPQVYTLPPSREEMTKNQVSLTCLVKGFYP | 47 |
| chain | SDIAVEWESNGQPENNYKTTPPVLDSDGSFFLYS | |
| | KLTVDKSRWQQGNVFSCSVMHEALHNHYTQKS | |
| | LSLSPGK | |
| constant region of mouse | AKTTAPSVYPLAPVCGDTTGSSVTLGCLVKGYFPEPV | 48 |
| IgG2a heavy chain | TLTWNSGSLSSGVHTFPAVLQSDLYTLSSSVTVTSST | |
| | WPSQSITCNVAHPASSTKVDKKIEPRGPTIKPCPPCK | |
| | CPAPNLLGGPSVFIFPPKIKDVLMISLSPIVTCVVVD | |
| | VSEDDPDVQISWFVNNVEVHTAQTQTHREDYNSTLRV | |
| | VSALPIQHQDWMSGKEFKCKVNNKDLPAPIERTISKP | |
| | KGSVRAPQVYVLPPPEEEMTKKQVTLTCMVTDFMPED | |
| | IYVEWTNNGKTELNYKNTEPVLDSDGSYFMYSKLRVE | |
| | KKNWVERNSYSCSVVHEGLHNHHTTKSFSRTPGK | |
| hinge area of constant region | EPRGPTIKPCPPCKCP | 49 |
| of mouse IgG2a heavy chain | | |
| CH1 of constant region of | AKTTAPSVYPLAPVCGDTTGSSVTLGCLVKGYFPEPV | 50 |
| mouse IgG2a heavy chain | TLTWNSGSLSSGVHTFPAVLQSDLYTLSSSVTVTSST | |
| | WPSQSITCNVAHPASSTKVDKKI | |
| CH2 + CH3 of constant region | APNLLGGPSVFIFPPKIKDVLMISLSPIVTCVVVDVS | 51 |
| of mouse IgG2a heavy chain | EDDPDVQISWFVNNVEVHTAQTQTHREDYNSTLRVVSA | |
| | LPIQHQDWMSGKEFKCKVNNKDLPAPIERTISKPKGS | |
| | VRAPQVYVLPPPEEEMTKKQVTLTCMVTDFMPEDIY | |
| | VEWTNNGKTELNYKNTEPVLDSDGSYFMYSKLRVEK | |
| | KNWVERNSYSCSVVHEGLHNHHTTKSFSRTPGK | |
| CL (constant region | RADAAPTVSIFPPSSEQLTSGGASVVCFLNNFYPKDI | 52 |
| of mouse kappa light chain) | NVKWKIDGSERQNGVLNSWTDQDSKDSTYSMSSTLTL | |
| | TKDEYERHNSYTCEATHKTSTSPIVKSFNRNEC | |

### EXAMPLES

### Example 1. Anti-CLDN18.2 Monoclonal Antibodies and Confirmation of Binding Capacity and Specificity of the Anti-CLDN18.2 Monoclonal Antibodies to CLDN18.2 Protein or CLDN18.2 Expressing Cells

1. Lead Murine Anti-Human CLDN18.2 Antibodies and Humanized Anti-CLDN18.2 Antibodies

We selected three lead murine anti-human CLDN18.2 antibodies (m-anti-human CLDN18.2-6 (also referred to as m-6), m-anti-human CLDN18.2-12 (also referred to as m-12), and m-anti-human CLDN18.2-13 (also referred to as m-13), and two humanized CLDN18.2 antibodies hCLDN18.2 808 and hCLDN18.2 841. VH and VL sequences thereof are listed in Table 1.

2. The Kinetics of CLDN18.2 Monoclonal Antibody m-13 Binding to hCLDN18.2 Protein were Measured Using a BIAcore 8K Instrument Based on Surface Plasmon Resonance (SRP) Technology.

VH and VL sequences of m-13 are shown in Table 1, the heavy chain constant region of m-13 is the constant region of mouse IgG2a heavy chain, and the light chain of m-13 is a mouse kappa light chain. The sequences thereof are listed in Table 4.

For kinetic measurements, hCLDN18.2 protein (Genscript) was serially diluted with HBS-EP⁺1× (cytiva, BR-1006-69) buffer, starting at 50 nM, with 4 concentration gradients of 2-fold dilution and set 0 concentration. Startup 3 times. m-13 antibody: 2 μg/mL, injection time 100 s, flow rate 10 μL/min, captured with ProA chip (cytiva, 29127556); antigen protein hCLDN18.2(Genscript): binding for 120 s, flow rate 30 μL/min, dissociation for 600 s; regeneration: regeneration with MgCl₂ buffer for 30 s, flow rate 30 μL/min.

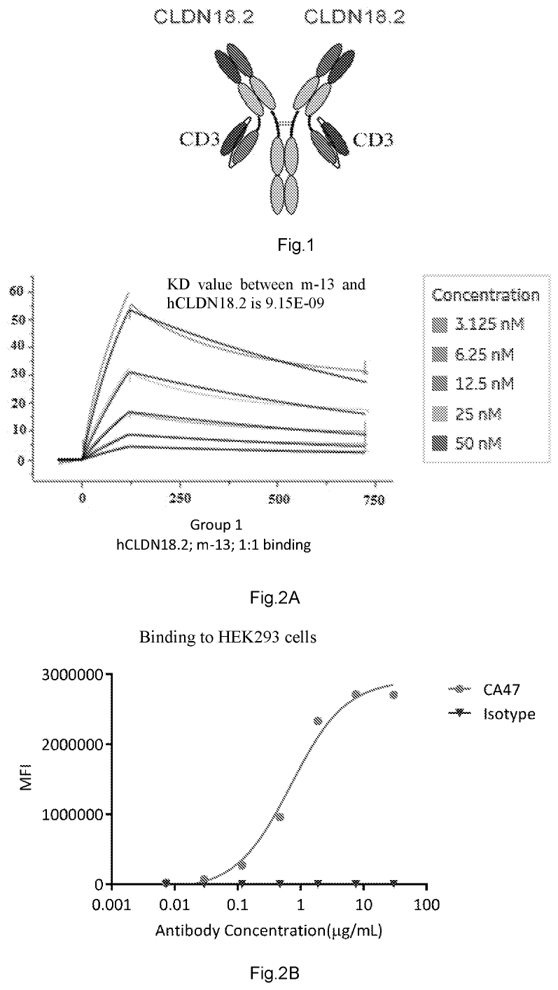

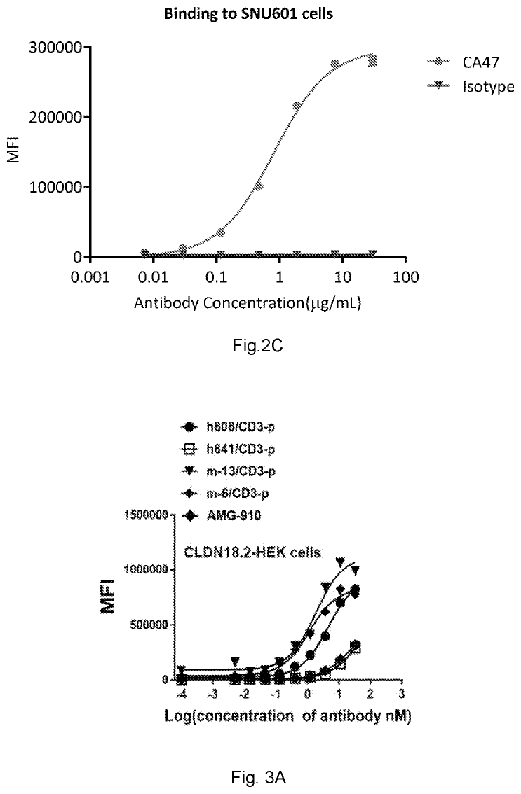

The binding constant (ka) and dissociation constant (kd) were calculated using the 1:1 binding model (BIAcore Evaluation Software version 3.2), and the Equilibrium Dissociation Constant (KD) was calculated as the ratio of kd/ka. The results can be seen in FIG. 2A, and the KD value between m-13 and hCLDN18.2 is 9.15E-09.

3. Confirmation of Binding Affinity of CLDN18.2 Monoclonal Antibody m-13 to CLDN18.2 Overexpressing HEK293 Cells and SNU601 Cells

3.1 We First Compared Binding Affinity of the CLDN18.2 Monoclonal Antibodies to Human CLDN18.2-Overexpressing HEK293 Cells.

To determine binding of CLDN18.2 monoclonal antibody on the cell surface; fifty thousand hCLDN18.2-expressing HEK293 cells (KYinno) per well were placed in a 96-well V-bottom plate (Corning) and incubated with test monoclonal antibody m-13 on ice for 30 minutes. HEK293 cells were washed with FACS buffer (PBS, 2% FBS, 1 mM EDTA) and stained with goat anti-human-IgG-PE Fab (Jackson Immuno Research) on ice for 30 minutes. HEK293 cells were washed twice with FACS buffer and DAPI was added to the final suspension, and the apparent binding activity was assessed by flow cytometry and the results are shown in FIG. 2B. IC50=0.701 μg/mL, and the Isotype used in FIG. 2B is an antibody for another target but has the same Fc with m-13.

3.2 We Further Compared Binding Affinity of the CLDN18.2 Monoclonal Antibodies to Human SNU601 Cells.

To determine binding of CLDN18.2 monoclonal antibody on the cell surface; fifty thousand SNU601 cells (Cobioer) per well were placed in a 96-well V-bottom plate (Corning) and incubated with test monoclonal antibody m-13 on ice for 30 minutes. SNU601 cells were washed with FACS buffer (PBS, 2% FBS, 1 mM EDTA) and stained with goat anti-human-IgG-PE Fab (Jackson Immuno Research) on ice for 30 minutes. SNU601 cells were washed twice with FACS buffer and DAPI was added to the final suspension, and the apparent binding activity was assessed by flow cytometry and the results are shown in FIG. 2C. IC50=0.811 μg/mL, and the Isotype used in FIG. 2C is an antibody for another target but has the same Fc with m-13.

### Example 2. Generation of CLDN18.2/CD3 Bispecific Antibodies and Evaluation of the Bispecific Antibodies

1. Generation of CLDN18.2/CD3 Bispecific Antibodies and Evaluation of the Bispecific Antibodies and Lead CLDN18.2/CD3 Bispecific Antibodies Candidate Selection

1.1. Generation of CLDN18.2/CD3 Bispecific Antibodies

The above two lead humanized CLDN18.2 antibodies (hCLDN18.2 808 and hCLDN18.2 841) and three lead murine anti-human CLDN18.2 antibodies (m-anti-human CLDN18.2-6 (also referred to as m-6), m-anti-human CLDN18.2-12 (also referred to as m-12), and m-anti-human CLDN18.2-13 (also referred to as m-13) were used to develop a new CLDN18.2/CD3 bispecific antibody (bispecific antibody against human CLDN18.2 and CD3) for CLDN18.2 positive tumor therapy. Humanized anti-CD3 antibody: SP34(i.e. CD3-p or CD3 Parental), CD3-v1, or CD3-v2 were used as one arm to construct CLDN18.2/CD3 bispecific antibody in a butterfly format (FIG. 1).

In this butterfly format, Fab of anti-CLDN18.2 antibody is used to bind CLDN18.2 expressed on tumor cells and two anti-CD3 scFv-arms fused to the C-terminus of the light chains of anti-CLDN18.2 is used to bind to CD3 on T cells. This format is bivalent for CLDN18.2 and functionally monovalent for CD3. Specifically, for the bispecific antibodies, anti-CLDN18.2 clone variable regions (VH and VL) were fused to the constant regions of human IgG1 heavy chain and human kappa light chain respectively; the sequences of the constant regions are listed in Table 4. The anti-CD3 single-chain variable fragment (scFv) is fused to the C terminus of the light chains of anti-CLDN18.2. To avoid Ig Fc mediated interactions, mutations L234A, L235A and P329A were introduced in the human IgG1 Fc region to eliminate effector functions (e.g. ADCC/CDC function). CLDN18.2/CD3 bispecific antibody with conventional Fc (e.g. Fc of IgG1, IgG2, IgG3 or IgG4) can also mediate killing effect.

1.2. Evaluation of Binding Affinity of the CLDN18.2/CD3 Bispecific Antibodies (i.e. CLDN18.2/CD3 bsAbs) to Human CLDN18.2-Overexpressing HEK293 Cells

We first compared binding affinity of the CLDN18.2/CD3 bsAbs to human CLDN18.2-overexpressing HEK293 cells.

To determine binding of CLND18.2/CD3 bispecific antibodies on the cell surface; twenty thousand hCLND18.2-overexpressing HEK293 cells(ATCC, CRL-3216) per well were placed in a 96-well V-bottom plate (Corning) and incubated with test bsAbs (h808/CD3-p, h841/CD3-p, m-6/CD3-p, m-13/CD3-p, or AMG-910) on ice for 30 minutes. HEK293 cells were washed with FACS buffer (PBS, 2% FBS, 1 mM EDTA) and stained with goat anti-human-IgG-PE Fab (Jackson Immuno Research) on ice for 30 minutes. Cells were washed twice with FACS buffer and DAPI was added to the final suspension. The apparent binding activity was assessed by flow cytometry and the results are shown in FIG. 3A, hCLDN18.2 808/CD3 (also referred to as h-808/CD3-p) and two murine CLDN18.2/CD3-p (m-6/CD3-p and m-13/CD3-p) had comparable binding affinity to CLDN18.2, and higher binding affinity to CLDN18.2 than hCLDN18.2 841/CD3-p (also referred to h841/CD3-p) and benchmark bispecific antibody Amgen CLDN18.2/CD3 (AMG-910) (the amino acid sequence depicted in SEQ ID NO: 132 of US20200055932A1).

1.3. Evaluation of Binding Affinity of the CLDN18.2/CD3 Bispecific Antibodies to CD3⁺ Jurkat Cells

To compare CD3 binding affinity of Amgen CLDN18.2/CD3(AMG-910) bsAb with CLDN18.2/CD3 bsAbs having mutant anti-CD3 arm (h841/CD3-v1 and h841/CD3-v2) or having parental anti-CD3 arm (h841/CD3-p), CD3⁺ Jurkat cells(ATCC, Clone E6-1) were stained with h841/CD3-p, h841/CD3-v1 as well as h841/CD3-v2 with both CD3-v1 and CD3-v2 having reduced CD3 binding affinity. As shown in FIG. 3B, AMG-910 bsAb showed a similar binding affinity to CD3 as h841/CD3-p bsAb having parental anti-CD3 arm (also referred to as CD3-p), but much higher binding affinity than our h841/CD3-v1 and h841/CD3-v2 having mutant anti-CD3 arm (i.e. CD3-v1 and CD3-v2) (FIG. 3B) (VH and VL sequences of CD3-p, CD3-v1 and CD3-v2 are listed in Table 3).

1.4. Evaluation of TCR-NFAT Activity Mediated by CLDN18.2/CD3 bsAb in NFAT-Reporter Jurkat Cells

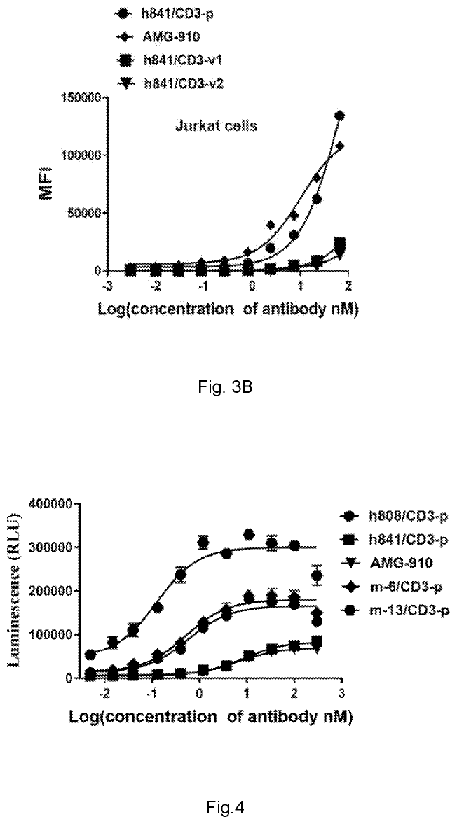

We further verified TCR-NFAT activity mediated by CLDN18.2/CD3 bsAb in NFAT-reporter Jurkat cells (InvivoGen, jktl-nfat). NFAT activity mediated by AMG-910 bsAb and h841/CD3-p was significantly lower than NFAT activity mediated by h808/CD3-p, m-6/CD3-p, and m-13/CD3-p (FIG. 4), even though h808/CD3-p, h841/CD3-p and AMG-910 had similar binding affinity to CD3. This finding indicated that TCR signaling strength triggered by CLDN18.2/CD3 bsAbs depends on binding affinity of CLDN18.2/CD3 bsAb to CD3 as well as the target molecule CLDN18.2.

1.5. Evaluation of T Cells Killing Effect Mediated by CLDN18.2/CD3 bsAb in Three Tumor Cell Lines with Various Expression Levels of hCLDN18.2

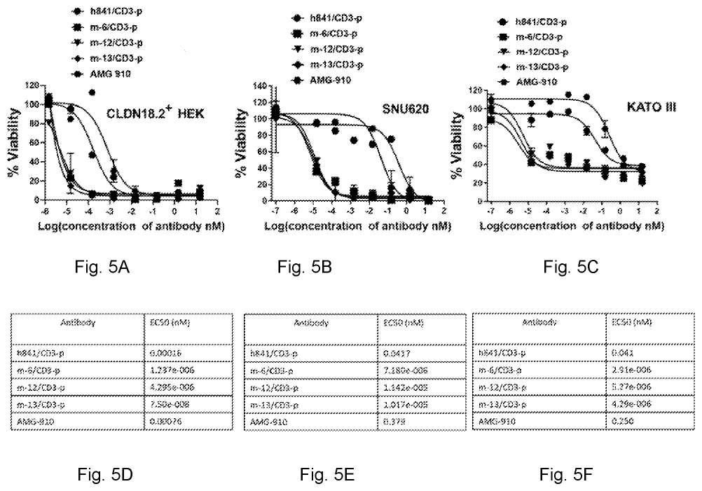

In selecting a lead candidate antibody, one of the most important functions is whether it can efficiently mediate T cells to kill targets. To evaluate this, we chose three tumor cell lines with various expression levels of hCLDN18.2 as targets. Freshly isolated human PBMCs (STEMCELL Technologies) were cocultured with the target cells in serial dilution of the tested CLDN18.2/CD3 bsAbs at E/T ratio of 20:1 for 48 hours. Killing activity to the target cells was determined by luminescence units as these target cells were transfected with luciferase. As shown in FIG. 5A-5C, three murine CLDN18.2/CD3 bsAbs (i.e. mCLDN18.2/CD3 bsAbs): m-6/CD3-p, m-12/CD3-p, and m-13/CD3-p displayed better killing activity against high CLDN18.2 expressing HEK (ATCC, CRL-3216) (i.e. CLDN18.2⁺HEK) (FIG. 5A), medium CLDN18.2 expressing SNU620 (Korean Cell Line Bank, 00620.1) (FIG. 5B), and low CLDN18.2 expressing KATO III (ATCC, HTB-103) (FIG. 5C) target cells as compared to h841/CD3-p and AMG-910 BsAb. The differences in killing potency between mCLDN18.2/CD3 bsAbs and AMG-910 were even larger against the medium (SNU620) (FIG. 5B) and low (KATO III) expressing CLDN18.2 cells (FIG. 5C), which suggests that mCLDN18.2/CD3 bsAbs have significant potential development value with a better therapeutic index than AMG-910. FIG. 5D-5F shows EC50 of killing potency for murine CLDN18.2/CD3 bispecific antibodies.

1.6. Evaluation of Cytokines Release Mediated by CLDN18.2/CD3 bsAb

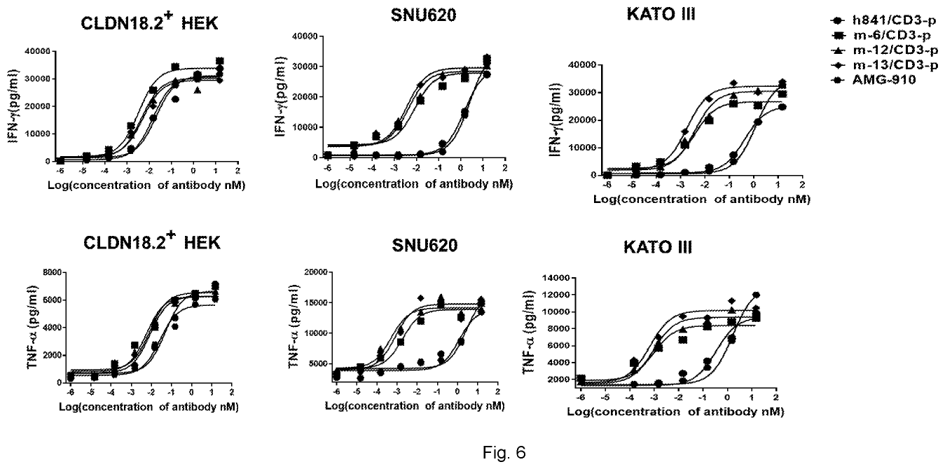

T-cell engager antibody can mediate direct cell-cell contacts between CTLs and target cells, which results in killing target cells. On the other hand, activated T cells can simultaneously secrete cytokines, such as IFN-α and TNF-γ as long as TCR stimulation continues by antibody engagement. These cytokines affect the target cell or cell distal to the effector T cells. TNF-α engages its receptor on the target cell and triggers target-cell apoptosis, and IFN-γ increases Fas-mediated target-cell lysis. However, although cytokines are necessary for the functioning of T cells, high cytokine release levels mediated by T cell engager antibody would induce a potential unfavorable cytokine release syndrome (CRS). Therefore, proper levels of cytokine release mediated by CLDN18.2/CD3 bispecific antibodies would need to be taken into consideration for evaluation of a lead candidate. For this reason, a parallel study was designed to investigate cytokine release in the same co-culture condition as the killing assay. The target cells and human PBMCs were plated in 96-well microplates at an E:T ratio of 20:1 and co-cultured with various concentrations of anti-CLDN18.2/CD3 bsAbs(h841/CD3-p, AMG-910, m-6/CD3-p, m-12/CD3-p, and m-13/CD3-p). After 48 hours of incubation, cell-free supernatants were harvested, and the production of INF-α, TNF-γ, IL-2, and IL-10 secretion was measured by ELISA in the supernatants from the coculture of PBMCs with various CLDN18.2 expressing HEK (i.e. CLDN18.2 HEK), SNU620, and KATO III target cells. In line with cytolytic activity, m-6/CD3-p, m-12/CD3-p, and m-13/CD3-p clones induced higher INF-α (FIG. 6), TNF-γ (FIG. 6), IL-2, and IL-10 release (data not shown). By contrast, lower levels of cytokine release were observed from h841/CD3-p and AMG-910.

1.7. Evaluation of Binding Specificity of Anti-CLDN18.2 Antibodies and Lead CLDN18.2/CD3 Bispecific Antibodies Candidate Selection

As CLDN18.1 and CLDN18.2 share highly homologous sequences, the assessment of binding specificity of anti-CLDN18.2 antibodies is critical for the development of therapeutic Claudin18.2 bispecific antibodies. From the killing assays (FIG. 5A-5C), we found that the three murine anti-human CLDN18.2 (m-6, m-12, and m-13) CD3 bsAbs displayed more potent cytotoxicity than h841/CD3 against various CLDN18.2 expressing targets. Therefore, binding specificity of those CLDN18.2 antibodies needed to be assessed against CLDN18.1 expressing cell lines.

As described above, we tested the specificity of h808/CD-3-p, h841/CD3-p, and three murine CLDN18.2/CD3-p BsAbs on human CLDN18.1-overexpressing CHO cells (Creative Biogene, SCS-R00629). In general, after dissociating cells and washing in PBS, 1×10⁵ CLDN18.1 expressing CHO cells were seeded in a 96 well plate. The anti-CLDN18.2/CD3 antibodies prepared in a final concentration of 25 μg/mL were incubated with cells for 1 hour at 4° C. After washing with FACS wash buffer, plates were incubated with PE conjugated goat anti-Human IgG, Fc Fragment Specific antibody (1:200 diluted in FACS wash buffer) for 20 minutes at 4° C. Mean fluorescence intensity (MFI) was measured using NovoCyte 2060 and results were analyzed by GraphPad software.

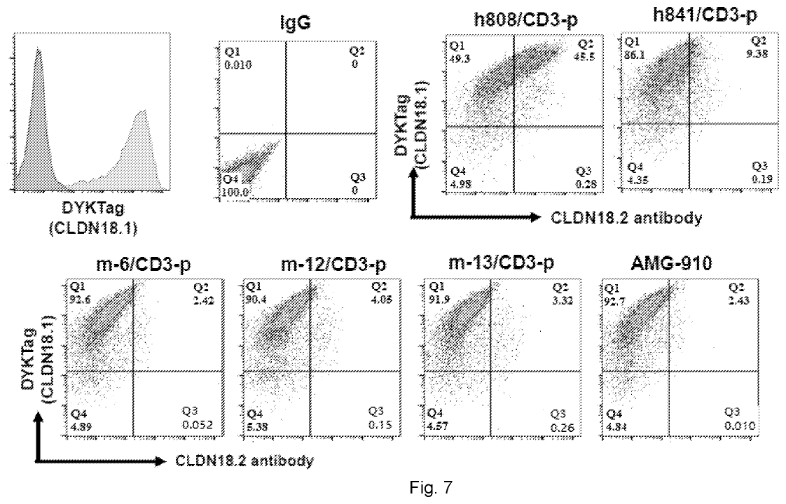

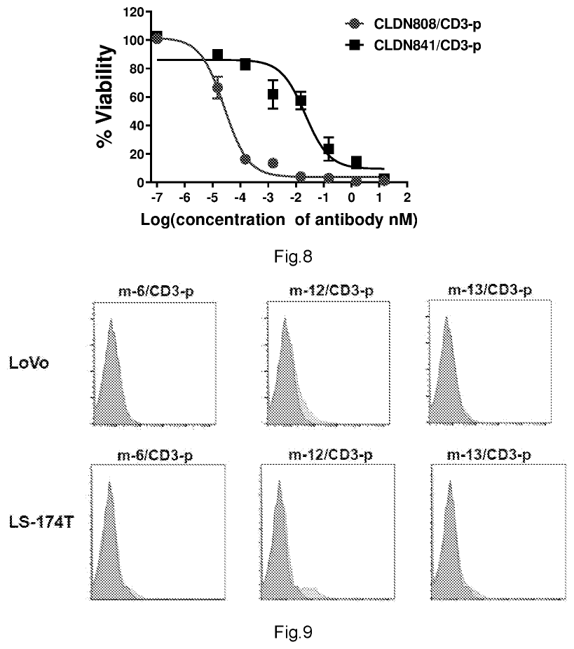

As shown in FIG. 7, m-6/CD3-p, m-12/CD3-p, m-13/CD3-p, h841/CD3-p and AMG-910, exhibit clear binding specificity and no cross-reactivity with human CLDN18.1-overexpressing CHO. Unexpectedly, the data from flow cytometry assays showed h808/CD3-p had high cross-reactivity with CLDN18.1. As a result, this cross-reactivity led to a devaluation of h808/CD3-p bispecific antibody as a lead candidate for further development even though it displayed very potent cytotoxicity (FIG. 8). In addition, h841/CD3-p did not show binding to CLDN18.1; however, lower binding affinity and poor killing potency to target cells were disadvantages of utilizing h841/CD3-p (FIG. 8).

To further confirm binding specificity of the murine CLDN18.2 clones m-6/CD3-p, m-12/CD3-p, m-13/CD3-p were tested against CLDN18.2 negative tumor cell lines LoVo (ACTT, CCL-229) and LS-174T (ATCC, LC-188) using flow cytometry assays. As shown in FIG. 9, m-6/CD3-p and m-13/CD3-p were not found to bind to the tested CLDN18.2 negative tumor cells, while m-12/CD3-p showed weak and non-specific binding to LoVo and LS-174T cells (FIG. 9).

2. Evaluation of Humanized CLDN18.2-13/CD3 Bispecific Antibodies

2.1 Binding Affinity of CLDN18.2-13/CD3 Bispecific Antibodies

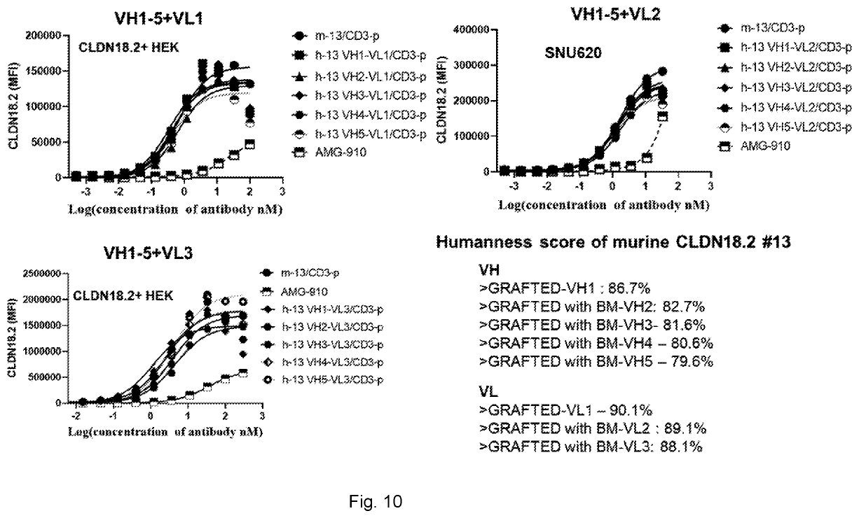

As m-13 and m-6 clones displayed high binding affinity, high specificity, and potent cytotoxicity, m-13 was finally chosen as a lead candidate for humanization and m-6 also was humanized as a back-up clone. The VH and VL sequences of humanized m-6 (i.e., h-6) are shown as SEQ ID Nos. 29-30 listed in Table 2-1, respectively. For m-13, the top 3 humanized variable light chains and 5 variable heavy chains were obtained, which reached high human-ness scores (VL1-90.1%, VL2-89.1%, VL3-88.1%, VH1-86.7%, VH2-82.7%, VH3-81.6%, VH4-80.6%, and VH5-79.6%) (VH and VL sequences are listed in Table 2). To characterize the CLDN18.2-13 antibodies (i.e. CLDN18.2-h-13 antibodies) resulting from those humanized heavy chains (i.e. VH1-VH5) and light chains (i.e. VL1-VL3), each humanized light chain was co-transfected with each humanized CLDN18.2-13 heavy chain in CD3 bispecific format. As a result, 15 humanized CLDN18.2-13/CD3 bispecific antibodies were generated. To determine their apparent binding activity, high CLDN18.2 expressing HEK293 and SNU620 cell lines as targets were incubated in varying concentrations of humanized CLDN18.2/CD3 bispecific antibodies, and with anti-human IgG-PE as a secondary antibody. The cells were then washed twice with FACS buffer. FACS analysis was carried out on a FACS Flow Cytometer. Through flow cytometry assays, we characterized the binding affinity of humanized CLDN18.2-13/CD3 subclones to CLDN18.2 on hCLDN18.2 over-expressing HEK293 cells (high expression of CLDN18.2) and CLDN18.2 natural expressing SNU-620 cells (medium expression of CLDN18.2). We found that all 15 humanized CLDN18.2-13/CD3 candidates had comparable binding affinities as parental m-13 (FIG. 10) as judged by median fluorescence intensity (MFI) on both CLDN18.2 highly expressing HEK cells as well as CLDN18.2 medium expressing SNU620 cells.

2.2 High Specificity of CLDN18.2-13/CD3 Bispecific Antibodies

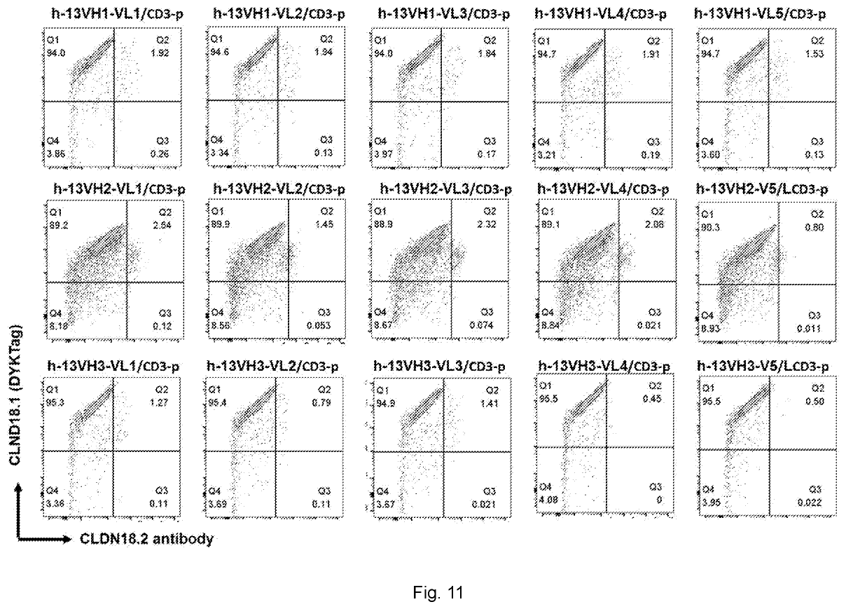

Although all humanized CLDN18.2-13/CD3 bsAbs retained high binding affinity to CLDN18.2 similar to parental m-13, whether the humanization of m-13 clone would change binding specificity was also very important to determine for the resultant humanized heavy chains and light chains. Therefore, we examined binding specificity of humanized CLDN18.2-13/CD3 subclones by staining human CLDN18.1 overexpressing CHO cells. As shown in FIG. 11, all 15 humanized CLDN18.2-13 subclones had no binding to CLDN18.1 on the transfected CHO cells, which suggests that humanized CLDN18.2 heavy and light chains retained binding specificity to CLDN18.2 similar to their parental m-13.

2.3 TCR/CD3 Signaling Strength Mediated by CLDN18.2-13/CD3 Bispecific Antibodies (i.e. h-13/CD3 bsAbs) with CD3-p (i.e. CD3 Parental) Arm.

In addition to binding affinity, TCR/CD3 signaling strength mediated by humanized CLDN18.2-13/CD3 subclones was also validated using coculture of NFAT-luciferase Jurkat cells with CLDN18.2⁺ SNU620 cells for 6 hours. To do so, 1×10⁵ Jurkat NFAT-luciferase reporter cells and 1×10⁵ CLDN18.2-expressing NSU620 tumor cells were seeded in a 96-well plate and incubated in 100 μL media+10% FBS and a serial dilution of anti-CEA/CD3 bsAbs at 37° C. for 6 hours. Bio-Glo™ Reagent (Promega) was added and luminescence was quantified using the BioMax Discover system. The data were fitted to a 4PL curve using GraphPad Prism 8 software.

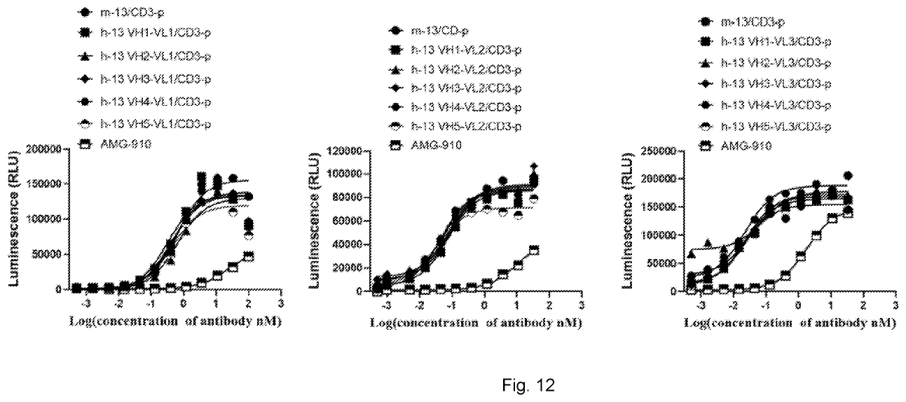

NFAT activity mediated by the 15 humanized CLDN18.2-13/CD3-p subclones, m-13/CD3-p, and Amgen CLDN18.2/CD3 bsAbs (AMG-910) were compared based on the luminescence readout. As expected, humanized CLDN18.2-13/CD3-p subclones induced a similar level of NFAT activity as their parental m-13/CD3-p (FIG. 12).

2.4 Anti-Tumor Effect of CLDN18.2-13/CD3 Bispecific Antibodies (i.e. h-13/CD3 bsAbs) with CD3-p (i.e. CD3 Parental) Arm

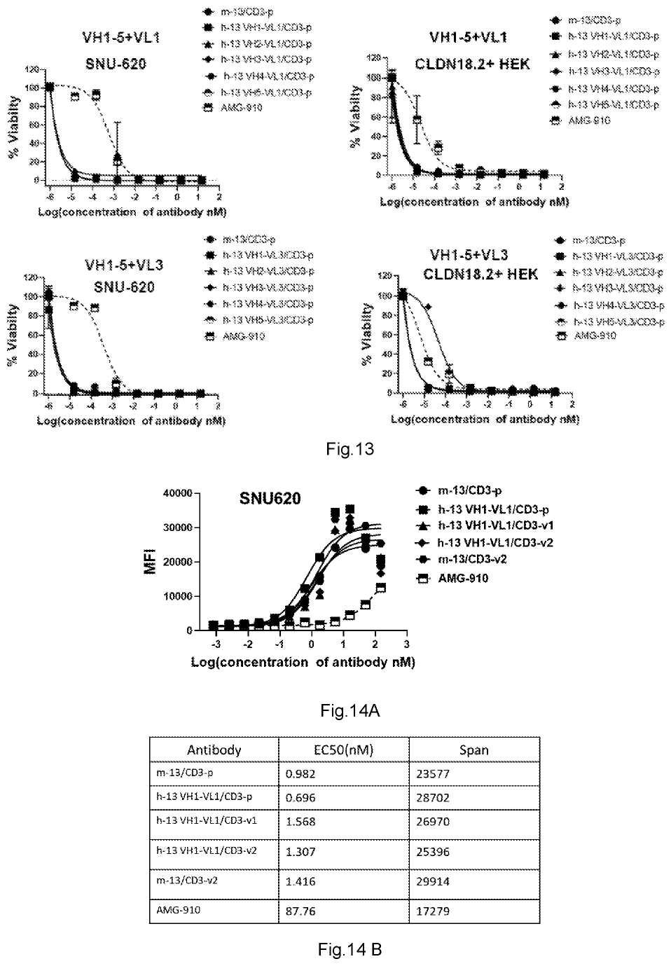

Finally, we compared the anti-tumor effect of humanized CLDN18.2-13/CD3 subclones (i.e.h-13/CD3-p subclones) with m-13/CD3-p. CLDN18.2 over-expressing HEK/Luc cells (i.e. CLDN18.2⁺HEK) (ATCC, CRL-3216) and medium CLDN18.2 expressing SNU620 cells were cocultured with PBMCs in serial dilutions of the tested antibodies at a final E/T ratio of 20:1. Specific lysis of tumor cells was determined after a 48-hour coculture by quantification of luciferase intensity units. As shown in FIG. 13, the humanized CLDN18.2-13/CD3 subclones induced potent T cell redirected cytotoxicity against their targets. As expected, the killing potency mediated by humanized CLDN18.2-13/CD3 subclones and parental m-13/CD3-p was 100-fold higher than that of AMG-910 (FIG. 13). Taken together, these functional assays indicated that the murine CLDN18.2 clone 13 has been successfully humanized.

2.5 Binding Affinity of CLDN18.2-13/CD3 Bispecific Antibodies (i.e. h-13/CD3 bsAbs) with Mutant CD3 Arm (CD3-V1 or CD3-V2) to CLDN18.2 and CD3

Since h-13/CD3-p carried SP34 as a CD3 arm, which has high binding affinity to CD3, potential cytokine release syndrome (CRS) may be induced in the clinic. To generate safe and effective CLDN18.2/CD3 bispecific antibody and to reduce the potential CRS related toxicity, we used our optimized CD3 platform, which includes several CD3 binders with various binding affinities. Our previous project has already demonstrated that the usage of the lower binding CD3 binders as arms for construction of CEA/CD3 bispecific antibodies can significantly reduce cytokine release and still retain a potent killing activity.

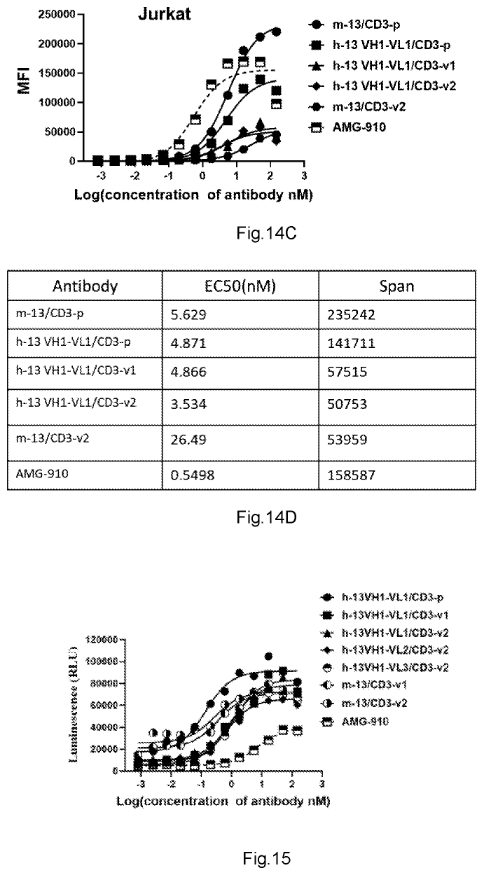

Therefore, the humanized CLDN18.2-13 heavy chain 1 (VH1) was used to co-transfect CHO cells with h-13 light chains (VL1) linked with the CD3-P, or mutant CD3 binders (CD3-V1 and CD3-V2) (sequences are listed in Table 3). Eventually, three h-13/CD3 variant bispecific antibodies were generated, h-13VH1-VL1/CD3-p, h-13VH1-VL1/CD3-v1, and h-13VH1-VL1/CD3-v2. As the mutant CD3 scFv was linked to hCLDN18.2 light chain, we first examined whether binding affinity of h-13/CD3-v1 and h-13/CD3-v2 to CLDN18.2 on the cell surface would be affected. Therefore, CLDN18.2 positive SNU620 cells were stained with h-13/CD3 variant bispecific antibodies by FACS assay. As shown in FIG. 14A, all h-13/CD3 subclones had comparable binding affinity to CLDN18.2 as their parental m-13 binders (i.e.m-13/CD3-p). FIG. 14B shows EC50 of binding affinity of humanized CLDN18.2/CD3 bispecific antibodies compared with murine parental CLDN18.2-13/CD3 bispecific antibody.

To confirm binding of CD3 variants to CD3 on T cells, we performed a Jurkat binding assay. As we expected, FACS analysis showed that h-13/CD3-v1 and h-13/CD3-v2 displayed significantly reduced binding affinities compared to h-13/CD3-p with parental CD3 binders (i.e.m-13/CD3-p) (FIG. 14C). FIG. 14D shows EC50 of binding affinity of humanized CLDN18.2/CD3 bispecific antibodies compared with murine parental CLDN18.2-13/CD3 bispecific antibody.

2.6 TCR Signaling Mediated by of CLDN18.2-13/CD3 Bispecific Antibodies (i.e. h-13/CD3 bsAbs) with Mutant CD3 Arm (CD3-V1 or CD3-V2)

We then further evaluated the strength of TCR signaling mediated by h-13/CD3 bsAbs by NFAT reporter assay. As described in section 2.3, Jurkat NFAT-luciferase reporter cells and CLDN18.2-expressing NSU620 tumor cells were seeded in a 96-well plate and incubated for 6 hours. Luminescence was quantified using the BioMax Discover system.

Consistent with their binding affinity (FIG. 14C), three h-13/CD3-v2 subclones and the h-13/CD3-v1 subclone induced lower NFAT activity as compared to h-13/CD3-p in coculture of Jurkat-NFAT reporter cells with SNU620 target cells (FIG. 15). However, there is still higher NFAT activity mediated by h-13/CD3-v1 and h-13/CD3-v2 subclones than that of Amgen 18.2/CD3 bsAb (i.e. AMG-910) that carried a higher CD3 binding affinity binding arm, which once again suggested that binding affinity to CLDN18.2 also plays an important role in the T cell engagers.

2.7 Cytolytic Potency Mediated by CLDN18.2-13/CD3 Bispecific Antibodies (i.e. h-13/CD3 bsAbs) with Mutant CD3 Arm (CD3-V1 or CD3-V2)

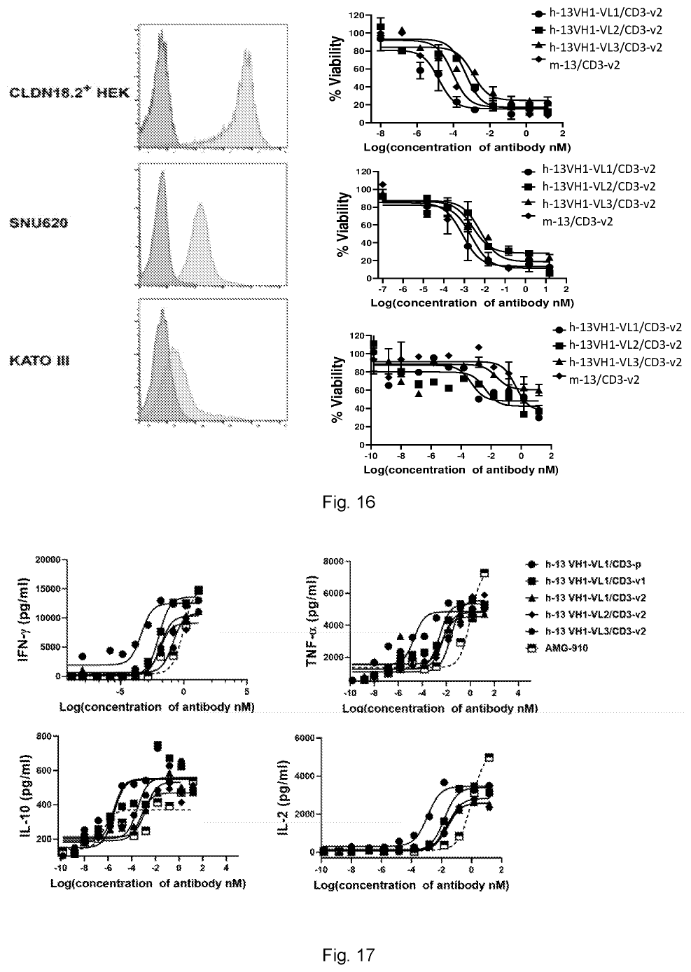

As CLDN18.2-13/CD3-v2 has lower binding affinity to CD3, which will significantly reduce cytokine release, three CLDN18.2/CD3-v2 bispecific antibodies with VH1 plus different VL1-3 chains were generated. As a result, we further compared cytolytic potency mediated by the 3 h-13/CD3 subclones using various CLDN18.2-expressing HEK (i.e. CLDN18.2⁺HEK), SNU620 and KATO III cells as a tumor target (FIG. 16 left panel). Interestingly, h-13VH1-VL1/CD3-v2 showed the highest potency against various CLDN18.2 expressing targets among the m-13/CD3-v2 clone and humanized h-13/CD3-v2 subclones (FIG. 16 right panel) in spite of similar binding and NFAT activity as the other h-13/CD3-v2 subclones (FIG. 15).

2.8 Cytokine Release Level Mediated by CLDN18.2-13/CD3 Bispecific Antibodies (i.e. h-13/CD3 bsAbs) with Mutant CD3 Arm (CD3-V1 or CD3-V2)

Moreover, since cytokine release level is usually correlated with killing potency, we further compared the cytokine release from the supernatants of SNU620 and PBMCs coculture system.

As described in Section 1.6, the supernatants of SNU620 and PBMC coculture experiments were collected for cytokine release measurement after 48 hours. IFN-γ, TNF-α, IL-10, and IL-2 release were quantified using BD optEIA human IFNγ and TNFα ELISA kits. Briefly, ELISA plates were coated with capture antibodies overnight. The supernatants were diluted at 1:20 with ELISA buffer and detector antibodies were added after 2 hour-incubation. ELISA substrate was added and the plates were read at 450 nM in SpectraMax reader after HRP was added into each well. Interestingly, h-13VH1-VL1/CD3-v2 induced comparable IFN-γ, TNF-α, IL-10, and IL-2 release as the other two h-13/CD3-v2 subclones (FIG. 17). Most importantly, h-13/CD3-v2 subclones mediated significantly lower cytokine release as compared to h-13/CD3-p, which was clearly driven by the higher CD3 binding affinity of h-13/CD3-p. Taken together, h-13VH1-VL1/CD3-v2 was selected as the lead candidate antibody based on its higher killing potency and similar cytokine release as compared to the other two h-13/CD3-v2 subclones.

1. A CLDN18.2 antibody or antigen binding fragment thereof, comprising a heavy chain variable region and a light chain variable region, wherein the heavy chain variable region comprising HCDR1 comprises the amino acid sequence represented by SEQ ID NO. 11, HCDR2 comprising the amino acid sequence represented by SEQ ID NO. 12 and HCDR3 comprising the amino acid sequence represented by SEQ ID NO. 13, and/or the light chain variable region comprising LCDR1 comprises the amino acid sequence represented by SEQ ID NO. 14, LCDR2 comprising the amino acid sequence represented by SEQ ID NO. 15 and LCDR3 comprising the amino acid sequence represented by SEQ ID NO. 16.

2. The CLDN18.2 antibody or antigen binding fragment of claim 1, wherein the antigen binding fragment is selected from scFv fragment, Fv fragment, F(ab′)2 fragment, Fab′-SH fragment and Fab′ fragment.

3. The CLDN18.2 antibody or antigen binding fragment thereof of claim 1, wherein the antibody is a monoclonal antibody and/or the antibody is a humanized antibody.

4. The CLDN18.2 antibody or antigen-binding fragment thereof of claim 1, wherein the antibody or antigen-binding fragment comprises a light chain constant domain, or the antibody or antigen-binding fragment comprises a light chain constant domain and a heavy chain constant domain.

5. A bispecific antibody, comprising a first antibody or antigen binding fragment that binds to CLDN18.2 or a variant thereof, and a second antigen binding domain that binds to CD3 or a variant thereof, wherein the first antibody or antigen binding fragment is the CLDN18.2 antibody or antigen-binding fragment of claim 1.

6. The bispecific antibody of claim 5, wherein the first antibody or antigen binding fragment comprises two identical heavy chains and two identical light chains, and wherein the second antigen binding domain comprises two identical single chain antibody fragments (scFv).

7. The bispecific antibody of claim 6, wherein each of the light chains of the first antibody or antigen binding fragment thereof is fused to each of the single chain antibody fragments (scFv) of the second antigen binding domain.

8. The bispecific antibody of claim 6, wherein the single chain antibody fragment (scFv) of CD3 comprises a heavy chain variable region and a light chain variable region, wherein
the heavy chain variable region comprises HCDR1 comprising the amino acid sequence represented by SEQ ID NO. 31, HCDR2 comprising the amino acid sequence represented by SEQ ID NO. 32 and HCDR3 comprising the amino acid sequence represented by SEQ ID NO. 33, and/or the light chain variable region comprises LCDR1 comprising the amino acid sequence represented by SEQ ID NO. 34, LCDR2 comprising the amino acid sequence represented by SEQ ID NO. 35 and LCDR3 comprising the amino acid sequence represented by SEQ ID NO. 36;
the heavy chain variable region comprises HCDR1 comprising the amino acid sequence represented by SEQ ID NO. 37, HCDR2 comprising the amino acid sequence represented by SEQ ID NO. 38 and HCDR3 comprising the amino acid sequence represented by SEQ ID NO. 39, and/or the light chain variable region comprises LCDR1 comprising the amino acid sequence represented by SEQ ID NO. 34, LCDR2 comprising the amino acid sequence represented by SEQ ID NO. 35 and LCDR3 comprising the amino acid sequence represented by SEQ ID NO. 36; or
the heavy chain variable region comprises HCDR1 comprising the amino acid sequence represented by SEQ ID NO. 37, HCDR2 comprising the amino acid sequence represented by SEQ ID NO. 40 and HCDR3 comprising the amino acid sequence represented by SEQ ID NO. 39, and/or the light chain variable region comprises LCDR1 comprising the amino acid sequence represented by SEQ ID NO. 34, LCDR2 comprising the amino acid sequence represented by SEQ ID NO. 35 and LCDR3 comprising the amino acid sequence represented by SEQ ID NO. 36.

9. The bispecific antibody of claim 8, wherein
the heavy chain variable region comprises the amino acid sequence that is at least about 95%, 96%, 97%, 98%, 99% or 100% identical to the amino acid sequence represented by SEQ ID NO. 25, and a light chain variable region comprises the amino acid sequence that is at least about 95%, 96%, 97%, 98%, 99% or 100% identical to the amino acid sequence represented by SEQ ID NO. 26;
the heavy chain variable region comprises the amino acid sequence that is at least about 95%, 96%, 97%, 98%, 99% or 100% identical to the amino acid sequence represented by SEQ ID NO. 27, and a light chain variable region comprises the amino acid sequence that is at least about 95%, 96%, 97%, 98%, 99% or 100% identical to the amino acid sequence represented by SEQ ID NO. 26; or
the heavy chain variable region comprises the amino acid sequence that is at least about 95%, 96%, 97%, 98%, 99% or 100% identical to the amino acid sequence represented by SEQ ID NO. 28, and a light chain variable region comprises the amino acid sequence that is at least about 95%, 96%, 97%, 98%, 99% or 100% identical to the amino acid sequence represented by SEQ ID NO. 26.

10. An isolated nucleic acid comprising a nucleic acid sequence encoding the CLDN18.2 antibody or antigen binding fragment of claim 1.

11. A vector comprising the nucleic acid of claim 10.

12. An isolated host cell comprising the CLDN18.2 antibody or antigen binding fragment thereof of claim 1.

13. A pharmaceutical composition comprising the CLDN18.2 antibody or antigen binding fragment of claim 1.

14. A method of treating cancer in a subject in need thereof, comprising administering to the subject the pharmaceutical composition of claim 13.

15. The method of claim 14, wherein the cancer is a CLDN18.2-positive cancer.

16. The CLDN18.2 antibody or antigen binding fragment thereof of claim 3, wherein
the heavy chain variable region comprises the amino acid sequence that is at least about 95%, 96%, 97%, 98%, 99% or 100% identical to the amino acid sequence represented by SEQ ID NO. 9, and/or the light chain variable region comprises the amino acid sequence that is at least about 95%, 96%, 97%, 98%, 99% or 100% identical to the amino acid sequence represented by SEQ ID NO. 10; or
the antibody or antigen-binding fragment thereof is a humanized antibody or antigen-binding fragment thereof, the heavy chain variable region comprises the amino acid sequence that is at least about 95%, 96%, 97%, 98%, 99% or 100% identical to the amino acid sequence represented by any one of SEQ ID NOs. 17-21, and/or the light chain variable region comprises the amino acid sequence that is at least about 95%, 96%, 97%, 98%, 99% or 100% identical to the amino acid sequence represented by any one of SEQ ID NOs. 22-24.

17. The bispecific antibody of claim 7, wherein the C-terminal of the constant region of each of the light chains of the first antibody or antigen binding fragment is fused to the N-terminal of the heavy chain variable region of each of the single chain antibody fragments (scFv) of the second antigen binding domain directly or via a linker.

18. A pharmaceutical composition of claim 13, wherein the pharmaceutical composition further comprises one or more pharmaceutically acceptable excipients.

19. The method of claim 15, wherein the cancer is hematological cancer or solid cancer.

20. The method of claim 19, wherein the CLDN18.2-positive cancer is gastric cancer or pancreatic cancer.


SEQUENCE LISTING (52 sequences)

SEQ ID NO: 1 - protein, 125 aa, Artificial Sequence
  VH
QVQLVQSGAEVKKPGSSVKVSCKASGGTFSSYAISWVRQAPGQGLEWMGRIIPILGIANYAQKFQGRVTVTADKSTSTAYMELSSLRSEDTAVYYCARERDNWDPYYYYYGMDVWGQGTTVTVSS

SEQ ID NO: 2 - protein, 107 aa, Artificial Sequence
  VL
DIQMTQSPSTLSASVGDRVTITCRASQSINSWLAWYQQKPGKAPKLLIYKASSLESGVPSRFSGSGSGTEFTLTISSLQPDDFATYYCQQYNSFPLTFGGGTKVEIK

SEQ ID NO: 3 - protein, 124 aa, Artificial Sequence
  VH
EVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARELRFFDWLLGSAFDIWGQGTTVTVSS

SEQ ID NO: 4 - protein, 107 aa, Artificial Sequence
  VL
DIVMTQSPSSLSASVGDRVTITCRASQGIRNDLGWYQQKPGKAPKLLIYAASSLQSGVPSRFSGSGSGTDFTLTISSLQPEDFATYYCLQDYNYPRTFGQGTKVEIK

SEQ ID NO: 5 - protein, 118 aa, Artificial Sequence
  VH
EVQLVESGGGLVQPGGSLKLSCAASGFTFSSYGMSWVRQTPDKRLELVATINSNGGSTYYPDSVKGRFTISRDNAKNTLYLQMSSLKSEDTAMYYCARLSYGNSFANWGQGTLVTVSS

SEQ ID NO: 6 - protein, 113 aa, Artificial Sequence
  VL
DIQITQSPSSLTVTAGEKVTMSCQSSQSLLNSGNHRNYLTWYQQKPGQPPKLLMYWASTRESGVPDRFAGSGSGTDFTLTISSVQAEDLAIYYCQNTYYYPFTFGSGTKLEIK

SEQ ID NO: 7 - protein, 119 aa, Artificial Sequence
  VH
DVQLQQSGPELMKPGASVKISCKASDYSFTSYNMHWVKQSHGKSLEWIGYIDPFNGGTRYNQKFKGKATLTVDKSSSTAYMHISSLTSEDSAVYYCARGAYYGNSFDYWGQGTLVTVSS

SEQ ID NO: 8 - protein, 113 aa, Artificial Sequence
  VL
DIVMTQSPSSLTVTAGEKVTMNCKSSQSLLNSGNQKNYLTWYQQKPGQPPKLLNYWASTRESGVPDRFTGSGSGTDFTLTIGSVQAEDLAVYYCQNAYSFPFTFGSGTKLEIK

SEQ ID NO: 9 - protein, 118 aa, Artificial Sequence
  VH
QLQLQQSGTELVRPGSSVKISCKASGYAFSNYWMNWVKQRPGQGLEWIGQIYPGNGDTKYNGNFKGKATLTADKSSSTAYMQLSSLTSEDSADYFCARYYRGNCFDYWGQGTTLTVSS

SEQ ID NO: 10 - protein, 113 aa, Artificial Sequence
  VL
DIVLTQSPSSLTVTAGEKVTMSCKSSQSLLNSGNQKNYLTWYQQKPGQPPKLLIYWASTRDSGVPARFTGSGSGTDFTLTINSVQAEDLAVYYCQNAYTYPLTFGAGTKLEIK

SEQ ID NO: 11 - protein, 10 aa, Artificial Sequence
  CDR1
GYAFSNYWMN

SEQ ID NO: 12 - protein, 17 aa, Artificial Sequence
  CDR2
QIYPGNGDTKYNGNFKG

SEQ ID NO: 13 - protein, 9 aa, Artificial Sequence
  CDR3
YYRGNCFDY

SEQ ID NO: 14 - protein, 17 aa, Artificial Sequence
  CDR1
KSSQSLLNSGNQKNYLT

SEQ ID NO: 15 - protein, 7 aa, Artificial Sequence
  CDR2
WASTRDS

SEQ ID NO: 16 - protein, 9 aa, Artificial Sequence
  CDR3
QNAYTYPLT

SEQ ID NO: 17 - protein, 118 aa, Artificial Sequence
  VH1
QVQLVQSGAEVKKPGASVKVSCKASGYAFSNYWMNWVRQAPGQRLEWMGQIYPGNGDTKYNGNFKGRVTITRDTSASTAYMELSSLRSEDTAVYYCARYYRGNCFDYWGQGTLVTVSS

SEQ ID NO: 18 - protein, 118 aa, Artificial Sequence
  VH2
QVQLVQSGAEVKKPGASVKVSCKASGYAFSNYWMNWVRQAPGQGLEWIGQIYPGNGDTKYNGNFKGRVTITADTSASTAYMELSSLRSEDTAVYFCARYYRGNCFDYWGQGTLVTVSS

SEQ ID NO: 19 - protein, 118 aa, Artificial Sequence
  VH3
QVQLVQSGAEVKKPGASVKVSCKASGYAFSNYWMNWVKQAPGQGLEWIGQIYPGNGDTKYNGNFKGRVTITADTSASTAYMELSSLRSEDTAVYFCARYYRGNCFDYWGQGTLVTVSS

SEQ ID NO: 20 - protein, 118 aa, Artificial Sequence
  VH4
QVQLVQSGAEVKKPGASVKVSCKASGYAFSNYWMNWVKQAPGQGLEWIGQIYPGNGDTKYNGNFKGRVTLTADTSASTAYMELSSLRSEDTAVYFCARYYRGNCFDYWGQGTLVTVSS

SEQ ID NO: 21 - protein, 118 aa, Artificial Sequence
  VH5
QVQLVQSGAEVKKPGASVKVSCKASGYAFSNYWMNWVKQAPGQGLEWIGQIYPGNGDTKYNGNFKGRATLTADTSASTAYMELSSLRSEDTAVYFCARYYRGNCFDYWGQGTLVTVSS

SEQ ID NO: 22 - protein, 113 aa, Artificial Sequence
  VL1
DIVMTQSPDSLAVSLGERATINCKSSQSLLNSGNQKNYLTWYQQKPGQPPKLLIYWASTRDSGVPDRFSGSGSGTDFTLTISSLQAEDVAVYYCQNAYTYPLTFGGGTKVEIK

SEQ ID NO: 23 - protein, 113 aa, Artificial Sequence
  VL2
DIVMTQSPDSLAVSLGERATINCKSSQSLLNSGNQKNYLTWYQQKPGQPPKLLIYWASTRDSGVPDRFSGSGSGTDFTLTISSVQAEDVAVYYCQNAYTYPLTFGGGTKVEIK

SEQ ID NO: 24 - protein, 113 aa, Artificial Sequence
  VL3
DIVMTQSPDSLAVSLGERATMNCKSSQSLLNSGNQKNYLTWYQQKPGQPPKLLIYWASTRDSGVPDRFSGSGSGTDFTLTISSVQAEDVAVYYCQNAYTYPLTFGGGTKVEIK

SEQ ID NO: 25 - protein, 125 aa, Artificial Sequence
  VH
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 26 - protein, 109 aa, Artificial Sequence
  VL
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 27 - protein, 125 aa, Artificial Sequence
  VH
EVQLVESGGGLVKPGGSLKLSCAASGFTFSTYAMNWVRQAPGKGLEWVARIRSKYNNYATYYADSVKDRFTISRDDSKNTAYLQMNNLRTEDTAVYYCVRHGNWGNSYISYWAYWGQGTTVTVSS

SEQ ID NO: 28 - protein, 125 aa, Artificial Sequence
  VH
EVQLVESGGGLVKPGGSLKLSCAASGFTFSTYAMNWVRQAPGKGLEWVARIRSKYNNYATTYADSVKDRFTISRDDSKNTAYLQMNNLRTEDTAVYYCVRHGNWGNSYISYWAYWGQGTTVTVSS

SEQ ID NO: 29 - protein, 118 aa, Artificial Sequence
  VH
EVQLVESGGGLVQPGGSLRLSCAASGFTFSSYGMSWVRQAPGKGLEWVATINSNGGSTYYPDSVKGRFTISRDNAKNTLYLQMNSLRAEDTAVYYCARLSYGNSFANWGQGTTVTVSS

SEQ ID NO: 30 - protein, 113 aa, Artificial Sequence
  VL
DIVITQSPSSLSVTLGERATLSCRSSQSLLNSGNHRNYLTWYQQKPGQAPRLLMYWASTRESGVPDRFSGSGSGTDFTLTISSLQPEDFATYYCQNTYYYPFTFGGGTKLEIK

SEQ ID NO: 31 - protein, 5 aa, Artificial Sequence
  CDR1
KYAMN

SEQ ID NO: 32 - protein, 19 aa, Artificial Sequence
  CDR2
RIRSKYNNYATYYADSVKD

SEQ ID NO: 33 - protein, 14 aa, Artificial Sequence
  CDR3
HGNFGNSYISYWAY

SEQ ID NO: 34 - protein, 14 aa, Artificial Sequence
  CDR1
GSSTGAVTSGYYPN

SEQ ID NO: 35 - protein, 7 aa, Artificial Sequence
  CDR2
GTKFLAP

SEQ ID NO: 36 - protein, 9 aa, Artificial Sequence
  CDR3
ALWYSNRWV

SEQ ID NO: 37 - protein, 5 aa, Artificial Sequence
  CDR1
TYAMN

SEQ ID NO: 38 - protein, 19 aa, Artificial Sequence
  CDR2
RIRSKYNNYATYYADSVKD

SEQ ID NO: 39 - protein, 14 aa, Artificial Sequence
  CDR3
HGNWGNSYISYWAY

SEQ ID NO: 40 - protein, 19 aa, Artificial Sequence
  CDR2
RIRSKYNNYATTYADSVKD

SEQ ID NO: 41 - protein, 15 aa, Artificial Sequence
  Linker-1
GGGGSGGGGSGGGGS

SEQ ID NO: 42 - protein, 30 aa, Artificial Sequence
  Linker-2
GGGGSGGGGSGGGGSGGGGSGGGGSGGGGS

SEQ ID NO: 43 - protein, 109 aa, Artificial Sequence
  CL (constant region of human kappa light chain)
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTS

SEQ ID NO: 44 - protein, 98 aa, Artificial Sequence
  CH1 of human IgGl heavy chain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 45 - protein, 15 aa, Artificial Sequence
  Hinge
EPKSCDKTHTCPPCP

SEQ ID NO: 46 - protein, 110 aa, Artificial Sequence
  CH2 of human IgGl heavy chain
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAK

SEQ ID NO: 47 - protein, 107 aa, Artificial Sequence
  CH3 of human IgGl heavy chain
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 330 aa, Artificial Sequence
  constant region of mouse IgG2a heavy chain
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 49 - protein, 16 aa, Artificial Sequence
  hinge area of constant region of mouse IgG2a heavy chain
EPRGPTIKPCPPCKCP

SEQ ID NO: 50 - protein, 97 aa, Artificial Sequence
  CH1 of constant region of mouse IgG2a heavy chain
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKI

SEQ ID NO: 51 - protein, 217 aa, Artificial Sequence
  CH2+CH3 of constant region of mouse IgG2a heavy chain
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - protein, 107 aa, Artificial Sequence
  CL (constant region of mouse kappa light chain)
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC